(19) United States
(12) Patent Application Publication
(10) Pub. No.: US 2015/0272541 A1
(43) Pub. Date: Oct. 1, 2015

(54) ULTRASONIC MEASURING APPARATUS AND ULTRASONIC MEASURING METHOD

(71) Applicant: SEIKO EPSON CORPORATION, Tokyo (JP)

(72) Inventor: Takashi HYUGA, Matsumoto-shi (JP)

(21) Appl. No.: 14/674,083

(22) Filed: Mar. 31, 2015

(30) Foreign Application Priority Data
Apr. 1, 2014 (JP) 2014-075750
Apr. 7, 2014 (JP) 2014-078320
Dec. 19, 2014 (JP) 2014-257683

(51) Int. Cl. A61B 8/08; A61B 8/14

(52) U.S. Cl. CPC A61B 8/085; A61B 8/5207; A61B 8/14; A61B 8/5223; A61B 8/0891

(57) ABSTRACT

A set of feature points including three feature points in a B-mode image acquired by an ultrasonic measurement are evaluated on a criterion as to whether a position of a circle passing through the three feature points is regarded as a position of a vessel wall. As evaluation items, there are the number of feature points located on a contour of the circle passing through the three feature points, position changes of the feature points with time, luminance of the feature points, the number of feature points inside the circle, etc. As a result of the evaluation, if a predetermined condition is satisfied, the position of the vessel wall is determined to be in the position of the circle passing through the selected three feature points, and the vessel position is detected.

### CROSS-REFERENCE

The entire disclosure of Japanese Patent Application No. 2014-075750 filed on Apr. 1, 2014, No. 2014-257683 filed on Dec. 19, 2014, and No. 2014-078320 filed on Apr. 7, 2014 are expressly incorporated by reference herein.

### BACKGROUND 1. Technical Field

The present invention relates to an ultrasonic measuring apparatus that detects a vessel position using ultrasonic wave. 2. Related Art

As an example of a measurement of biological information using ultrasonic wave, evaluations of blood vessel functions including determination of a vascular disease are performed. For example, a measurement of blood pressure (fluctuations in vessel diameter), a measurement of IMT (Intima Media Thickness) of a carotid artery as an index of arteriosclerosis, an evaluation of hardness of a vessel wall, etc. are representative examples. In the measurements, first, a position of a vessel within a body tissue is measured.

As a specific technology of locating the vessel position, JP-A-2009-66268 discloses a technology of estimating and modeling a position and a shape of a carotid artery based on B-mode images as section images in the short-axis direction of the carotid artery. In the technology, with attention focused on movements of the artery due to heart beats, generation and optimization of an evaluation function of a model, and estimation and modeling of the position and the shape of the carotid artery of the next frame are repeated with respect to each frame.

In the technology disclosed in the above described JP-A-2009-66268, generation and optimization of the evaluation function and modeling are repeatedly performed for each frame, and there is a problem that calculation processing with respect to the measurement is complex and the amount of calculation increases. Further, for evaluation of the hardness of the carotid artery vessel wall, it is necessary to detect the position of the long axis of the vessel. Accordingly, it is important to detect the position of the vessel not only in the section images in the short-axis direction of the vessel but also in section images in the long-axis direction.

### SUMMARY

A first aspect of the invention relates to an ultrasonic measuring apparatus including an ultrasonic measurement unit that transmits and receives ultrasonic wave with respect to a vessel and acquires an ultrasonic image containing a section in a short-axis direction of the vessel, a feature point extraction unit that extracts feature points from the ultrasonic image, a combination selection unit that selects a combination of feature points in which positions of the feature points have a location relationship along a section shape of the vessel in the short-axis direction, and a position determination unit that determines a position of the vessel using the combination.

A second aspect of the invention relates to an ultrasonic measuring apparatus including an ultrasonic measurement unit that transmits and receives ultrasonic wave with respect to a vessel and acquires an ultrasonic image containing a section in a long-axis direction of the vessel, a feature point extraction unit that extracts feature points from the ultrasonic image, a combination selection unit that selects a combination of feature points in which positions of the feature points have a location relationship along a section shape of the vessel in the long-axis direction, and a position determination unit that determines a position of the vessel using the combination.

A third aspect of the invention relates to an ultrasonic measuring method of determining a vessel position from an ultrasonic image containing a section of a vessel in a short-axis direction or a long-axis direction using a computer, including extracting feature points from the ultrasonic image, selecting a combination of feature points in which positions of the feature points have a location relationship along a section shape of the vessel in the short-axis direction or the long-axis direction, and determining a position of the vessel using the combination.

### BRIEF DESCRIPTION OF THE DRAWINGS

The invention will be described with reference to the accompanying drawings, wherein like numbers reference like elements.

FIG. 1 is a system configuration diagram of an ultrasonic measuring apparatus in the first embodiment.

FIG. 2 is an explanatory diagram of detection of feature points in an ultrasonic image in the first embodiment.

FIG. 3 is an explanatory diagram of generation of a set of feature points in the first embodiment.

FIG. 4 is an explanatory diagram of an evaluation with respect to a first evaluation item in the first embodiment.

FIG. 5 is an explanatory diagram of an evaluation with respect to a second evaluation item in the first embodiment.

FIG. 6 is an explanatory diagram of an evaluation with respect to a third evaluation item in the first embodiment.

FIG. 7 shows an example of A-mode data in the first embodiment.

FIG. 8 is an explanatory diagram of an evaluation with respect to a fourth evaluation item in the first embodiment.

FIG. 9 is an explanatory diagram of an evaluation with respect to a fifth evaluation item in the first embodiment.

FIG. 10 is a functional configuration diagram of the ultrasonic measuring apparatus in the first embodiment.

FIG. 11 is a configuration diagram of a memory unit of the ultrasonic measuring apparatus in the first embodiment.

FIG. 12 is a flowchart of ultrasonic measurement processing in the first embodiment.

FIG. 13 shows a configuration example of a processing unit of an ultrasonic measuring apparatus in the second embodiment.

FIG. 14 shows a configuration example of a memory unit of the ultrasonic measuring apparatus in the second embodiment.

FIG. 15 is a flowchart showing a flow of vessel position determination processing in the second embodiment.

FIG. 16 is a flowchart showing a flow of anterior-posterior wall detection processing in the second embodiment.

FIGS. 17A to 17C are diagrams for explanation of the anterior-posterior wall detection processing in the second embodiment.

FIGS. 18A and 18B are diagrams for explanation of adventitia candidate point extraction processing in the second embodiment.

FIGS. 19A and 19B are diagrams for explanation of intima candidate point extraction processing in the second embodiment.

FIG. 20 is a flowchart showing a flow of center scanning line determination processing in the second embodiment.

FIG. 21 is a flowchart showing a flow of vessel position decision processing in the second embodiment.

FIGS. 22A to 22C are diagrams for explanation of a modified example of the adventitia candidate point extraction processing and the intima candidate point extraction processing in the second embodiment.

FIGS. 23A and 23B are diagrams for explanation of a modified example of the center scanning line determination processing in the second embodiment.

FIG. 24 is a system configuration diagram of an ultrasonic measuring apparatus in the third embodiment.

FIG. 25 is an explanatory diagram of detection of feature points in an ultrasonic image in the third embodiment.

FIG. 26 is an explanatory diagram of generation of a set of feature points in the third embodiment.

FIG. 27 is an explanatory diagram of an evaluation with respect to a first evaluation item in the third embodiment.

FIG. 28 is an explanatory diagram of an evaluation with respect to a second evaluation item in the third embodiment.

FIG. 29 is an explanatory diagram of an evaluation with respect to a third evaluation item in the third embodiment.

FIG. 30 shows an example of A-mode data in the third embodiment.

FIG. 31 is an explanatory diagram of an evaluation with respect to a fourth evaluation item in the third embodiment.

FIG. 32 is an explanatory diagram of an evaluation with respect to a fifth evaluation item in the third embodiment.

FIG. 33 is a functional configuration diagram of the ultrasonic measuring apparatus in the third embodiment.

FIG. 34 is a configuration diagram of a memory unit of the ultrasonic measuring apparatus in the third embodiment.

FIG. 35 is a flowchart of ultrasonic measurement processing in the third embodiment.

### DESCRIPTION OF EXEMPLARY EMBODIMENTS

According to the invention, a new technology of detecting a position of a vessel as an object of an ultrasonic measurement can be proposed.

An embodiment relates to an ultrasonic measuring apparatus including an ultrasonic measurement unit that transmits and receives ultrasonic wave with respect to a vessel and acquires an ultrasonic image containing a section in a short-axis direction of the vessel, a feature point extraction unit that extracts feature points from the ultrasonic image, a combination selection unit that selects a combination of feature points in which positions of the feature points have a location relationship along a section shape of the vessel in the short-axis direction, and a position determination unit that determines a position of the vessel using the combination.

Further, an embodiment relates to an ultrasonic measuring method of determining a vessel position from an ultrasonic image containing a section of a vessel in a short-axis direction using a computer, including extracting feature points from the ultrasonic image, selecting a combination of feature points in which positions of the feature points have a location relationship along a section shape of the vessel in the short-axis direction, and determining a position of the vessel using the combination.

According to the configurations, the combination of feature points in which the positions of the feature points in the ultrasonic image have the location relationship along the section shape of the vessel in the short-axis direction is selected and the position of the vessel is determined using the selected combination. There is a characteristic that many feature points appear along the contour of the section shape of the vessel in the ultrasonic image containing the section of the vessel in the short-axis direction. Thereby, a new technology of detecting the position of the vessel from the location relationship of the positions of the feature points in the ultrasonic image may be realized.

In the embodiment, the ultrasonic measuring apparatus wherein a contour position of a shape corresponding to the section of the vessel in the short-axis direction is estimated based on the location relationship with respect to the combination, probabilities of the contour position representing a position of a vessel wall of the vessel is calculated based on the contour position and the feature points, and thereby, the position of the vessel is determined may be formed.

According to the configuration, the contour position of the shape corresponding to the section of the vessel in the short-axis direction is estimated based on the location relationship of the positions of the feature points with respect to the combination, and the position of the vessel is determined using the probabilities of the contour position representing the position of the vessel wall. For example, attention is focused on many feature points appearing in the image part of the vessel wall in the ultrasonic image, and thereby, the position of the vessel wall may be detected.

In the embodiment, the ultrasonic measuring apparatus wherein a first of the probabilities is calculated using a number of the feature points located along the contour position may be formed.

According to the configuration, the first probability of the contour position representing the vessel wall is calculated using the number of the feature points located along the estimated contour position. In the ultrasonic image, many feature points appear in the image part of the vessel wall. The numbers of feature points in the contour position largely differ between the cases where the estimated contour position nearly coincides with the vessel wall and where not. Accordingly, the vessel position may be detected using the number of feature points in the estimated contour position.

In the embodiment, the ultrasonic measuring apparatus wherein a second of the probabilities is calculated using position changes of the feature points located along the contour position may be formed.

According to the configuration, the second probability of the contour position representing the vessel wall is calculated using the position changes of the feature points located along the estimated contour position. The vessel periodically repeats dilatation and constriction with beats and the positions of the feature points located on the vessel wall periodically change in synchronization with those, however, other body tissues than the vessel hardly move. That is, the position changes of the feature points with respect to the combinations largely differ between the cases where the estimated contour position nearly coincides with the vessel wall and where not. Accordingly, the vessel position may be determined using the position changes of the feature points.

In the embodiment, the ultrasonic measuring apparatus wherein a third of the probabilities is calculated using luminance of the feature points located along the contour position may be formed.

According to the configuration, the third probability of the contour position representing the vessel wall is calculated using the luminance of the feature points located along the estimated contour position. The reflectance of ultrasonic wave is higher on the vessel wall, and the luminance in the position of the vessel wall is higher in the ultrasonic image. Accordingly, the luminance of the feature points with respect to the combinations largely differs between the cases where the estimated contour position nearly coincides with the vessel wall and where not. Therefore, the vessel position may be determined using the luminance of the feature points.

In the embodiment, the ultrasonic measuring apparatus wherein a fourth of the probabilities is calculated using a number of the feature points located inside the contour position may be formed.

According to the configuration, the fourth probability of the contour position representing the vessel wall is calculated using the number of feature points located inside the estimated contour position. The reflectance of ultrasonic wave is extremely lower inside the vessel, and the feature points hardly appear inside the vessel. Accordingly, the vessel position may be determined using the number of feature points located inside the estimated contour position.

In the embodiment, the ultrasonic measuring apparatus wherein a fifth of the probabilities is calculated by comparison between a predetermined feature image that may be contained outside the vessel and an external image part of the contour position of the ultrasonic image may be formed.

According to the configuration, the fifth probability of the contour position representing the vessel wall is calculated by comparison between the external image part of the estimated contour position in the ultrasonic image and the predetermined feature image that may be contained outside the vessel.

In the embodiment, the ultrasonic measuring apparatus wherein the position determination unit determines a scanning line passing through a center of the vessel of a plurality of scanning lines with respect to transmission and reception of the ultrasonic wave using the combination may be formed.

According to the configuration, the scanning line passing through the center of the vessel maybe determined from the plurality of scanning lines with respect to transmission and reception of the ultrasonic wave using the selected combination of feature points.

In the embodiment, the ultrasonic measuring apparatus wherein the combination selection unit selects the combination of feature points in terms of scanning lines may be formed.

According to the configuration, the combination of feature points used for determination of the scanning line passing through the center of the vessel may be selected in terms of scanning lines.

In the embodiment, the ultrasonic measuring apparatus wherein the feature point extraction unit extracts adventitia positions and lumen-intima boundary positions with respect to an anterior wall and a posterior wall as feature points, and the position determination unit evaluates luminance of the respective feature points contained in the combination by a predetermined evaluation calculation, and specifies a scanning line with respect to the combination receiving a highest evaluation as a scanning line passing through the center of the vessel may be formed.

According to the configuration, the combination of the adventitia positions and the lumen-intima boundary positions with respect to the anterior wall and the posterior wall of the vessel may be used, the luminance thereof may be evaluated, and thereby, the scanning line passing through the center of the vessel may be specified.

In the embodiment, the ultrasonic measuring apparatus wherein the vessel is an artery may be formed.

According to the configuration, the position of the artery may be detected.

In the embodiment, the ultrasonic measuring apparatus further including a measurement unit that measures a predetermined vessel function of the vessel detected by the position determination unit may be formed.

According to the configuration, a series of processing of automatically finding a vessel and performing a vessel function measurement on the vessel may be realized.

Further, an embodiment relates to an ultrasonic measuring apparatus including an ultrasonic measurement unit that transmits and receives ultrasonic wave with respect to a vessel and acquires an ultrasonic image containing a section in a long-axis direction of the vessel, a feature point extraction unit that extracts feature points from the ultrasonic image, a combination selection unit that selects a combination of feature points in which positions of the feature points have a location relationship along a section shape of the vessel in the long-axis direction, and a position determination unit that determines a position of the vessel using the combination.

Furthermore, an embodiment relates to an ultrasonic measuring method of determining a vessel position from an ultrasonic image containing a section of a vessel in a long-axis direction using a computer, including extracting feature points from the ultrasonic image, selecting a combination of feature points in which positions of the feature points have a location relationship along a section shape of the vessel in the long-axis direction, and determining a position of the vessel using the combination.

According to the configurations, the combination of feature points in which the positions of the feature points in the ultrasonic image have the location relationship along the section shape of the vessel in the long-axis direction is selected and the position of the vessel is determined using the selected combination. There is a characteristic that many feature points appear in an image part of a vessel wall in the ultrasonic image containing the section of the vessel in the long-axis direction. Thereby, a new technology of detecting the position of the vessel from the location relationship of the feature points in the ultrasonic image may be realized. Obviously, the position of the long axis of the vessel can be detected from the ultrasonic image containing the section of the vessel in the long-axis direction.

In the embodiment, the ultrasonic measuring apparatus wherein a pair of straight lines corresponding to a section shape of the vessel in the long-axis direction is set in the ultrasonic image based on the location relationship with respect to the combination, probabilities of the pair of straight lines representing a position of a vessel wall of the vessel are calculated based on the pair of straight lines and the feature points, and the position of the vessel is determined using the probabilities and the combination may be formed.

According to the configuration, the pair of straight lines corresponding to the section shape of the vessel in the long-axis direction are set in the ultrasonic image based on the location relationship of the feature points with respect to the combination, and the position of the vessel wall is determined using the probabilities of the pair of straight lines representing the position of the vessel wall and the combination. For example, attention is focused on many feature points appearing in the image part of the vessel wall in the ultrasonic image, and thereby, the position of the vessel wall may be detected.

In the embodiment, the ultrasonic measuring apparatus wherein the calculation of the probabilities includes a calculation of a first of the probabilities using a number of the feature points located along the pair of straight lines may be formed.

According to the configuration, the first probability of the pair of straight lines representing the vessel wall is calculated using the number of the feature points located along the set pair of straight lines. In the ultrasonic image, many feature points appear in the image part of the vessel wall. The numbers of feature points in the position along the pair of straight lines largely differ between the cases where the set pair of straight lines nearly coincide with the vessel wall and where not. Accordingly, the vessel position may be detected using the number of feature points in the position along the pair of straight lines.

In the embodiment, the ultrasonic measuring apparatus wherein the calculation of the probabilities includes a calculation of a second of the probabilities using position changes of the feature points located along the pair of straight lines may be formed.

According to the configuration, the second probability of the position of the pair of straight lines representing the vessel wall is calculated using the position changes of the feature points located along the set pair of straight lines. The vessel periodically repeats dilatation and constriction with beats and the positions of the feature points located on the vessel wall periodically change in synchronization with those, however, other body tissues than the vessel hardly move. That is, the position changes of the feature points with respect to the combinations largely differ between the cases where the set pair of straight lines nearly coincide with the vessel wall and where not. Accordingly, the vessel position may be detected using the position changes of the feature points.

In the embodiment, the ultrasonic measuring apparatus wherein the calculation of the probabilities includes a calculation of a third of the probabilities using luminance of the feature points located along the pair of straight lines may be formed.

According to the configuration, the third probability of the position of the pair of straight lines representing the vessel wall is calculated using the luminance of the feature points located along the set pair of straight lines. The reflectance of ultrasonic wave is higher on the vessel wall, and the luminance in the position of the vessel wall is higher in the ultrasonic image. Accordingly, the luminance of the feature points with respect to the combinations largely differs between the cases where the set pair of straight lines nearly coincide with the vessel wall and where not. Therefore, the vessel position maybe detected using the luminance of the feature points.

In the embodiment, the ultrasonic measuring apparatus wherein the calculation of the probabilities includes a calculation of a fourth of the probabilities using a number of the feature points located between the pair of straight lines may be formed.

According to the configuration, as an evaluation with respect to the combination of feature points, the fourth probability of the position of the pair of straight lines representing the vessel wall is calculated using the number of feature points located between the set pair of straight lines. Inside the vessel, the reflectance of ultrasonic wave is extremely low and feature points hardly appear. Accordingly, the vessel position maybe detected using the number of feature points located between the estimated pair of straight lines.

In the embodiment, the ultrasonic measuring apparatus wherein the calculation of the probabilities includes a calculation of a fifth of the probabilities by comparison between a predetermined feature image that may be contained outside the vessel and an external image part of the pair of straight lines of the ultrasonic image may be formed.

According to the configuration, the fifth probability of the position of the pair of straight lines representing the vessel wall is calculated by comparison between the external image part of the pair of straight lines in the ultrasonic image and the predetermined feature image that may be contained outside the vessel.

In the embodiment, the ultrasonic measuring apparatus wherein the vessel is an artery may be formed.

According to the configuration, the position of the artery may be detected.

In the embodiment, the ultrasonic measuring apparatus further including a measurement unit that measures a predetermined vessel function of the vessel detected by the position determination unit may be formed.

According to the configuration, a series of processing of automatically finding a vessel and performing a vessel function measurement on the vessel may be realized.

As below, some embodiments to which the invention is applied will be explained. The forms to which the invention may be applied are not limited to the following embodiments.

### First Embodiment

### System Configuration

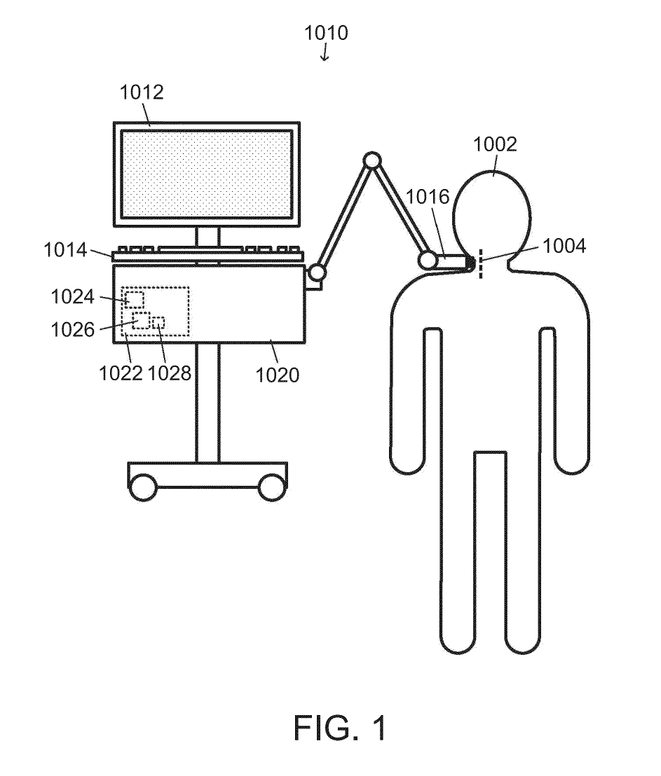

FIG. 1 shows a configuration example of an ultrasonic measuring apparatus 1010 in the first embodiment. The ultrasonic measuring apparatus 1010 is an apparatus that measures biological information of a subject using ultrasonic wave. In the embodiment, a vessel as a measuring object is a carotid artery and, as biological information, vessel function information such as IMT (Intima Media Thickness) is measured. Obviously, measurements of other vessel function information such as a measurement of vessel diameter and a measurement of blood pressure from the vessel diameter may be performed. Further, the vessel as the measuring object may be another artery such as a radial artery.

The ultrasonic measuring apparatus 1010 includes a touch panel 1012, a keyboard 1014, an ultrasonic probe 1016, a main body device 1020. A control board 1022 is mounted on the main body device 1020 and connected to the respective parts of the touch panel 1012, the keyboard 1014, the ultrasonic probe 1016, etc. so that signals can be transmitted and received.

On the control board 1022, a CPU (Central Processing Unit) 1024, an ASIC (Application Specific Integrated Circuit), various integrated circuits, a storage medium 1026 of an IC memory or a hard disk, and a communication IC 1028 that realizes data communication with an external device are mounted. The main body device 1020 executes control programs stored in the storage medium 1026 using the CPU 1024 etc., and thereby, realizes various functions including an ultrasonic measurement according to the embodiment.

Specifically, the main body device 1020 transmits and applies an ultrasonic beam toward an in vivo tissue of a subject 1002 from the ultrasonic probe 1016 and receives reflected wave. Then, the received signals of the reflected wave are amplified and signal-processed, and thereby, measurement data on the in vivo structure of the subject 1002 may be generated. The measurement data contains images of respective modes of the so-called A-mode, B-mode, M-mode, and color Doppler. The measurement using ultrasonic wave is repeatedly executed at a predetermined cycle. The unit of measurement is referred to as “frame”.

The ultrasonic probe 1016 includes a plurality of ultrasonic transducers arranged therein. In the embodiment, a single row is used, however, a two-dimensional arrangement configuration including a plurality of rows may be used. Further, the ultrasonic probe 1016 is fixed to the neck of the subject 1002 in the opposed position in which ultrasonic wave from the respective ultrasonic transducers may cross in the short-axis direction of a carotid artery (vessel) 1004 of the subject 1002, and the vessel function information is measured.

### Principle

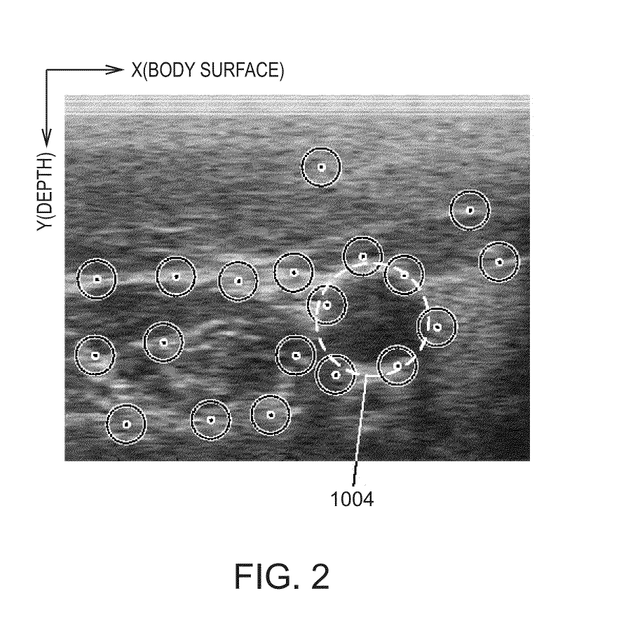

For measurement of the vessel function information, first, detection of a vessel position is performed. Specifically, as shown in FIG. 2, feature points in a B-mode image (center positions of dotted circles in FIG. 2) are extracted. Note that, to facilitate understanding, the reduced number of feature points is shown in the respective drawings of FIG. 2 and the subsequent drawings, however, actually, more feature points than those shown in the drawings are extracted. Further, in FIG. 2, a contour of the vessel 1004 is clearly shown by a broken line. The B-mode image shown in FIG. 2 is a sectional view of the vessel in the short-axis direction, and the X-axis extends along the living body surface and the Y-axis extends in the depth direction from the living body surface. As shown in FIG. 2, the section shape of the vessel 1004 in the short-axis direction is a nearly circular shape. Further, in the B-mode image, many of the feature points appear in parts in which luminance changes such as muscle, tendons, and fat in addition to the vessel walls (specifically, an intima-adventitia boundary and a lumen-intima boundary). The reflectance of ultrasonic wave is higher in the position where the medium changes, and the position with higher reflectance is represented in higher luminance in the B-mode image. Accordingly, the vessel wall, muscle, tendon, fat, etc. are different in medium from the surrounding tissues and the luminance changes in the parts, and the parts are extracted as feature points. One of the characteristics of the embodiment is in detection of the vessel position using the position relationship of the feature points.

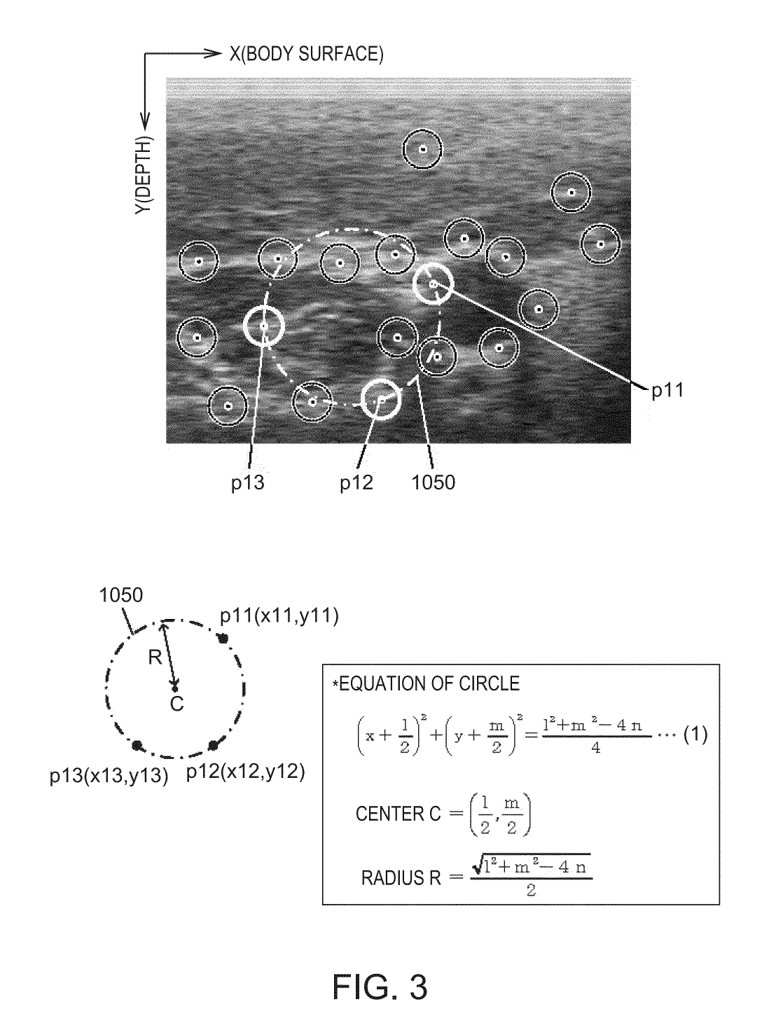

As shown in FIG. 3, three feature points (the center positions of solid white circles in FIG. 3) are selected from the extracted feature points in the B-mode image, and a set of feature points as a combination of the three feature points is generated. In this regard, the three feature points of the set of feature points may be randomly selected or selected so that the distances between one another maybe equal to or less than a predetermined distance without departing from the location relationship along the vessel wall.

Subsequently, with respect to the generated set of feature points, a circle passing through the respective feature points is obtained. That is, simultaneous linear equations with three unknowns are generated by substituting the respective position coordinates p11(x11, y11), p12(x12, y12), p13(x13, y13) of the three feature points into a general expression of a circle given by the formula (1), the simultaneous equations are solved, and thereby, parameters l, m, n defining the circle passing through the three feature points p11, p12, p13 are obtained and the contour position of the circle is estimated.

$${( x + \frac{1}{2} )}^{2} + {( y + \frac{m}{2} )}^{2} = \frac{{1}^{2} + {m}^{2} - 4 n}{4} \qquad ( 1 )$$

Namely, the positions of the three feature points forming the set of feature points have a location relationship along the contour of a circle 1050 (dashed-dotted circle in FIG. 3) as a shape corresponding to the short-axis section of the vessel. The circle 1050 having the contour position defined by the three feature points p11, p12, p13 forming the set of feature points is hereinafter referred to as “assumed circle 1050”.

Then, the set of feature points are evaluated on a criterion as to whether or not the counter of the corresponding assumed circle 1050 is regarded as the position of the vessel wall. Specifically, as expressed by the formula (2), evaluation values hi of the respective plurality of evaluation items are weighted by a coefficient ai and added, and a comprehensive evaluation value F is calculated.

$$F = {\sum }_{i} ( ai \times fi ) \qquad ( 2 )$$

Evaluation values fi of the respective evaluation items correspond to probabilities (also referred to as “accuracy”) of the contour of the assumed circle 1050 located in the position of the vessel wall. That is, the values are defined to be larger as the probabilities that the assumed circle 1050 is regarded as the vessel wall are larger. Further, whether or not the assumed circle 1050 of the set of feature points is regarded as the vessel wall is determined using the comprehensive evaluation value F, and the vessel position is decided. In this regard, setting of the evaluation items on which importance is placed for determination may be changed by the weight coefficient ai.

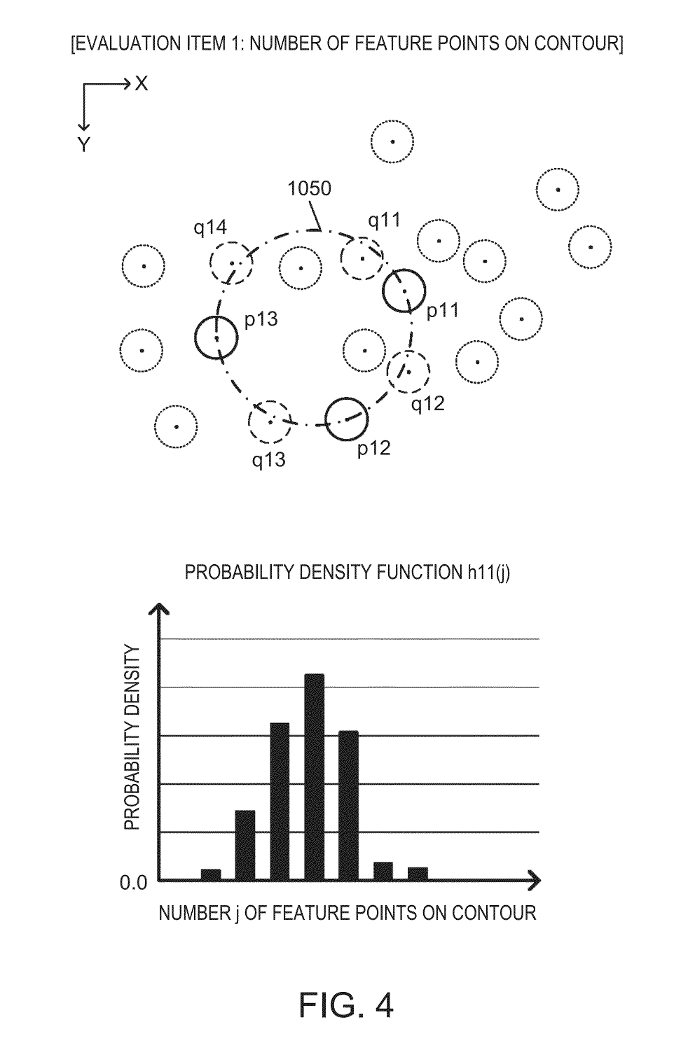

In the first embodiment, evaluations are made with respect to five evaluation items (first to fifth evaluation items). The first evaluation item is “number of feature points located on contour of assumed circle 1050 of set of feature points”. FIG. 4 is a diagram for explanation of evaluations with respect to the first evaluation item in the first embodiment. The upper side of FIG. 4 shows the schematic locations of the assumed circle 1050 and the feature points in the B-mode image and the lower side of FIG. 4 shows a probability density function h11(j) of the number of feature points as an evaluation criterion. The valuable j is the number of feature points.

As shown in FIG. 4, the feature points located at the distances from the contour of the assumed circle 1050 shown by the dashed-dotted line equal to or less than a predetermined short distance are selected as feature points q on the contour of the assumed circle 1050. Here, the feature points q do not include the feature points p11, p12, p13 forming the set of feature points. Then, the probability density obtained from the probability density function h11(j) based on the number j of the selected feature points q is used as the evaluation value f11 of the first evaluation item.

The probability density function h11(j) shown in FIG. 4 is defined, with respect to many B-mode images containing a vessel desired to be detected (e.g., a carotid artery) acquired in advance, by counting the numbers of feature points located on the vessel wall of the vessel. As described above, many feature points exist in the position of the vessel wall and there is a tendency that the numbers of the feature points are concentrated on predetermined numbers as shown by the probability density function h11(j).

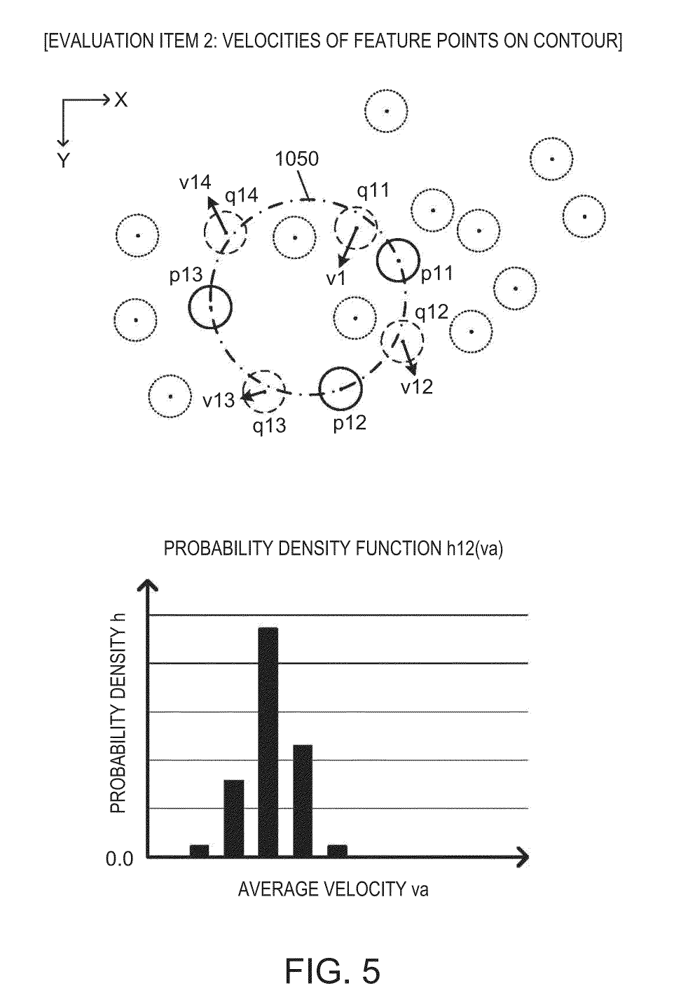

The second evaluation item is “displacement velocities of feature points located on contour of assumed circle 1050 of set of feature points”. The displacement velocity refers to a position change per unit time and a magnitude of the velocity (absolute value). FIG. 5 is a diagram for explanation of evaluations with respect to the second evaluation item in the first embodiment. The upper side of FIG. 5 shows the schematic locations of the assumed circle 1050 and the feature points in the B-mode image and the lower side of FIG. 5 shows a probability density function h12(va) of the average displacement velocity of feature points as an evaluation criterion. The variable va is an average velocity.

As shown in FIG. 5, the displacement velocities of the respective feature points q on the contour of the assumed circle 1050 are obtained, and average velocities va as averages of the displacement velocities are obtained. For example, the displacement velocities of the respective feature points q are obtained by obtaining velocity vectors v of the feature points q and averaging the magnitudes of the velocity vectors v over a predetermined period (equal to or more than one heartbeat of a heartbeat period, about several seconds) using a gradient method utilizing spatial luminance gradients, block matching utilizing an image block having a predetermined size and containing the feature points as a template, or the like. Then, the probability density obtained from the probability density function h12(va) in FIG. 5 based on the obtained average velocity va is used as an evaluation value f12 of the second evaluation item.

The probability density function h12(va) shown in FIG. 5 is defined, with respect to many B-mode images containing a vessel desired to be detected (e.g., a carotid artery) acquired in advance, by obtaining average velocities of the respective feature points located on the vessel wall of the vessel. The vessel repeats generally isotropic constriction and dilatation according to the beats of the heart. That is, the magnitude of the displacement velocity of the feature point located on the vessel wall periodically changes in terms of the heartbeat period, and the average in one heartbeat period is nearly constant in any heartbeat period. Accordingly, there is a tendency that the average values of the magnitudes of the displacement velocities of one heartbeat period are concentrated on predetermined values as shown by the probability density function h12(va).

Note that, as the displacement velocities of the respective feature points, velocity components in the depth direction (i.e., components of velocity vectors v in the depth direction) may be used. Further, not the displacement velocity, but acceleration may be used.

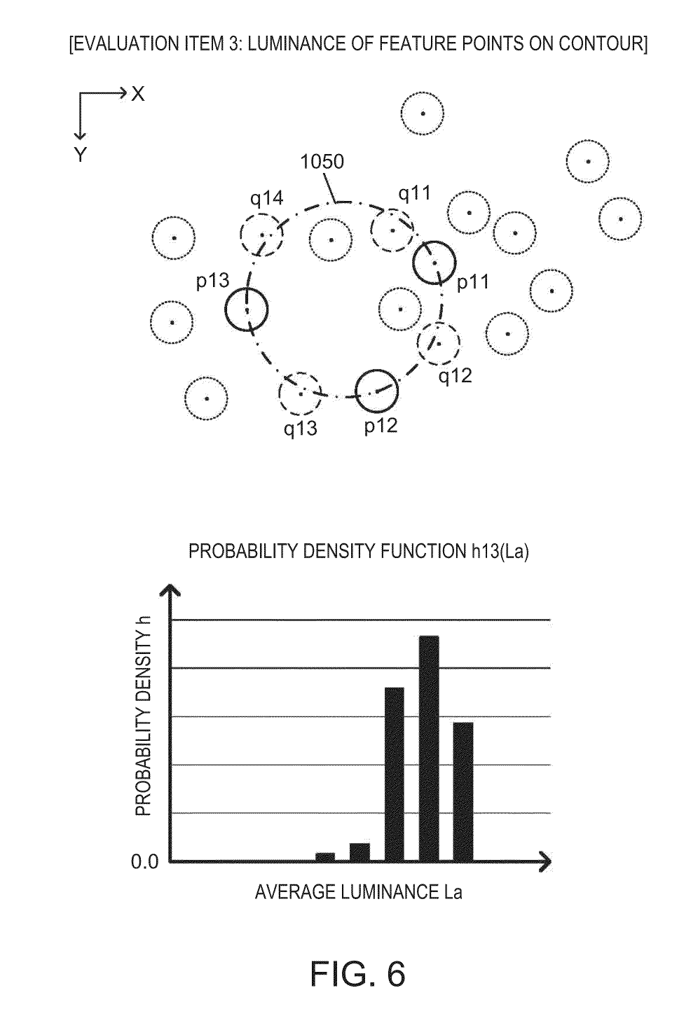

The third evaluation item is “luminance of feature points located on contour of assumed circle 1050 of set of feature points”. FIG. 6 is a diagram for explanation of evaluations with respect to the third evaluation item in the first embodiment. The upper side of FIG. 6 shows the schematic locations of the assumed circle 1050 and the feature points in the B-mode image and the lower side of FIG. 6 shows a probability density function h13(La) of the luminance as an evaluation criterion. The variable La is average luminance.

As shown in FIG. 6, average luminance La as averages of the luminance L of the respective feature points q is obtained. Then, the probability density obtained from the probability density function h13(La) based on the obtained average luminance La is used as an evaluation value f13 of the third evaluation item.

The probability density function h13(La) shown in FIG. 6 is defined, with respect to many B-mode images containing a vessel desired to be detected (e.g., a carotid artery) acquired in advance, by obtaining average values of luminance of feature points located on the vessel wall of the vessel. As described above, many feature points exist on the vessel wall, and there is a tendency that the average luminance of the feature points is concentrated on predetermined relatively high luminance as shown by the probability density function h13(La).

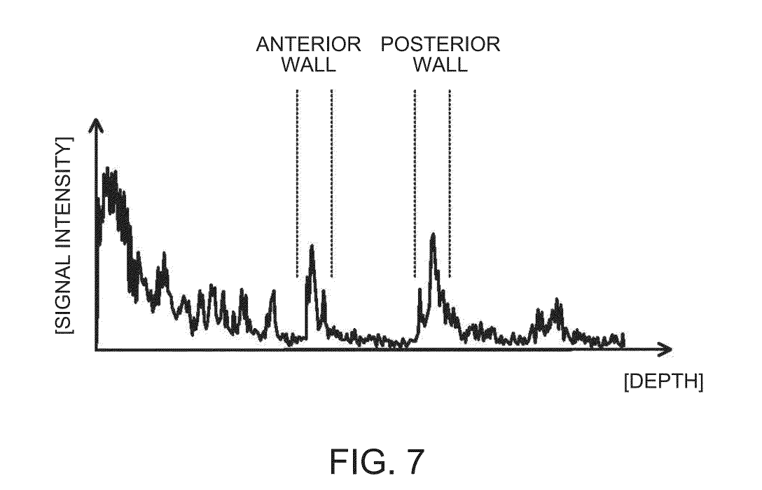

Note that, not the luminance of the feature points itself, but “gradient of luminance” may be used. That is, as shown in FIG. 7, in A-mode data (depth-signal intensity graph), signal intensity (i.e., luminance) largely changes in the depth position of the vessel wall. Thereby, as the gradients of luminance of the feature points q located on the contour of assumed circle 1050, gradients in the depth positions of the feature points q in the A-mode data (changes in signal intensity as seen in the depth direction) may be obtained, and the probability density may be obtained based on the average value of the gradients of luminance and used as the evaluation value of the third evaluation item. Further, as the gradients of luminance of the feature points q, differences between the luminance of the feature points q in the B-mode image and the luminance of pixels adjacent to the feature points q in the depth direction may be used.

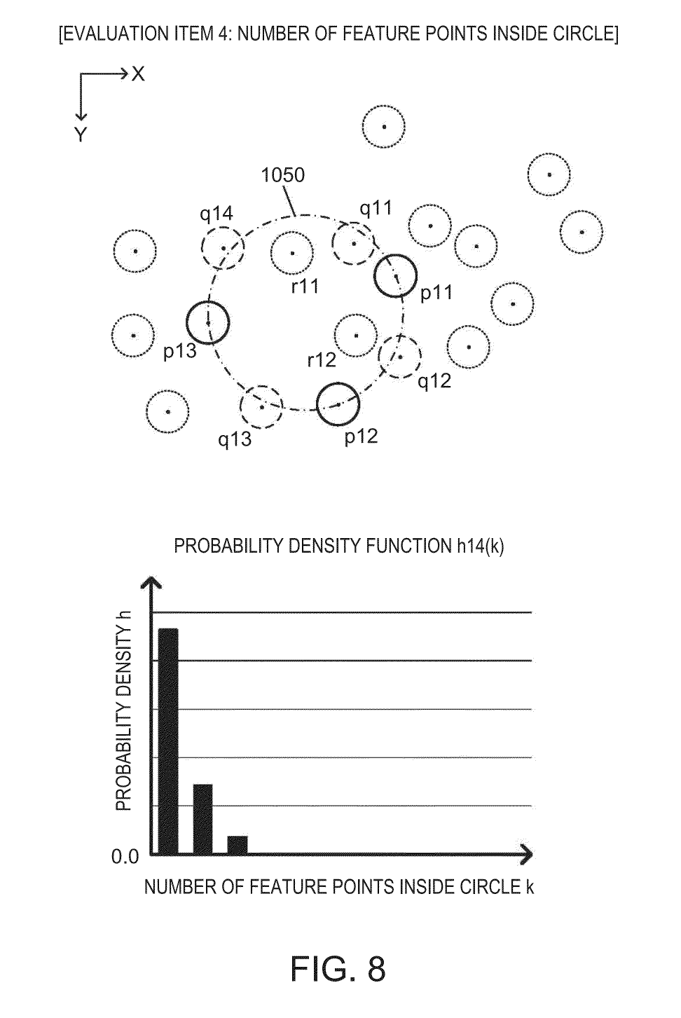

The fourth evaluation item is “number of feature points inside assumed circle 1050 of set of feature points”. FIG. 8 is a diagram for explanation of evaluations with respect to the fourth evaluation item in the first embodiment. The upper side of FIG. 8 shows the B-mode image and the lower side of FIG. 8 shows a probability density function h14(k) of the number of feature points as an evaluation criterion. The variable k is the number of feature points.

As shown in FIG. 8, feature points r located inside the assumed circle 1050 are selected. The feature points r selected here do not include the feature points p11, p12, p13 forming the set of feature points or the feature points q on the contour of the assumed circle as the evaluation objects in the first evaluation item. Then, the probability density obtained from the probability density function h14(k) based on the number k of the selected feature points r is used as an evaluation value f14 of the fourth evaluation item.

The probability density function h14(k) shown in FIG. 8 is defined, with respect to many B-mode images containing a vessel desired to be detected (e.g., a carotid artery) acquired in advance, by counting the numbers of feature points within the vessel (inside the vessel wall). The reflectance of ultrasonic wave by the vessel wall is higher, however, the reflectance by the blood within the vessel is extremely lower and the ultrasonic wave is hardly reflected, but transmitted. That is, there is a tendency that the numbers of feature points within the vessel are concentrated on predetermined numbers (values close to zero).

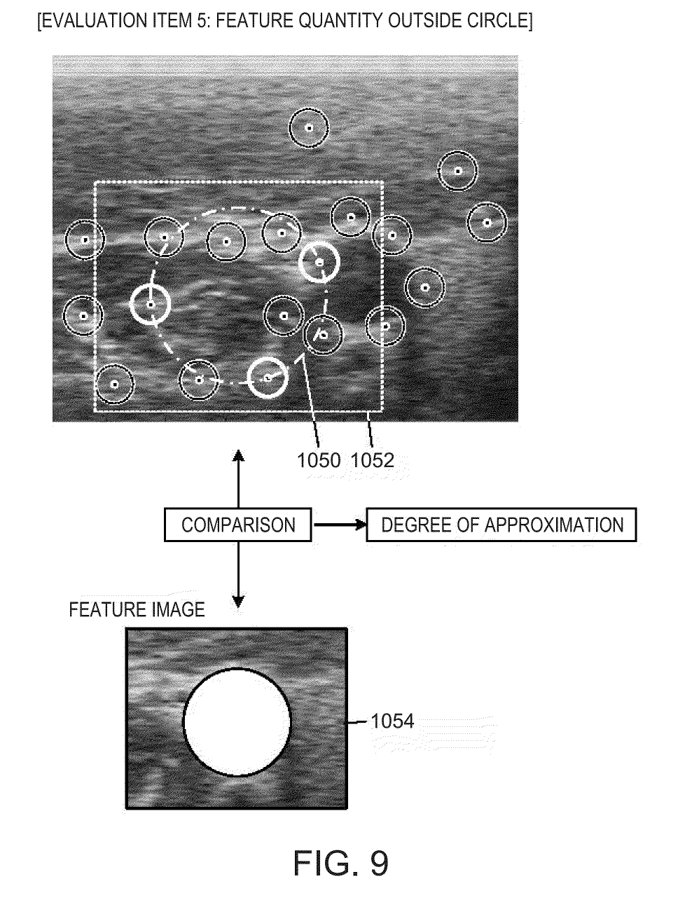

The fifth evaluation item is “feature quantity of external image of assumed circle”. FIG. 9 is a diagram for explanation of evaluations with respect to the fifth evaluation item in the first embodiment. As shown by the upper side of FIG. 9, a partial image 1052 in a predetermined range around the assumed circle 1050 is extracted as an evaluation object image from the B-mode image, feature quantity comparison processing between the evaluation object image and a feature image 1054 prepared in advance is performed, and a degree of approximation of the images is calculated. The degree of approximation is used as an evaluation value f15 of the fifth evaluation item.

More specifically, the partial image 1052 is the image obtained by setting the assumed circle 1050 in a predetermined position (e.g., at the center) and extracting a predetermined range based on the size of the assumed circle 1050 (e.g., a rectangular range formed by multiplying the diameter of the assumed circle 1050 by 1.5 in the longitudinal direction and by 2 in the lateral direction) from the B-mode image. The feature image 1054 has a white circle at the center and the relative position and the relative size of the circle to the whole feature image 1054 have the same relationship as that between the partial image 1052 and the assumed circle 1050.

The feature image 1054 is a B-mode image around the vessel desired to be detected (e.g., a carotid artery). Around the vessel, muscle fibers and groups of lymph nodes can exist as surrounding tissues, and the feature image 1054 contains pattern components of the surrounding tissues. In the feature quantity comparison processing, the outside part of the assumed circle 1050 is trimmed from the partial image 1052 (the inside part of the assumed circle 1050 is removed), a comparison calculation with the feature image 1054 is performed, and the degree of approximation is calculated. In the calculation of the degree of approximation, for example, the degree of approximation may be obtained by comparison between the location relationships of the feature points in the images, distributions of luminance, texture information of the images, or the like using the so-called pattern matching or the like.

### Functional Configuration

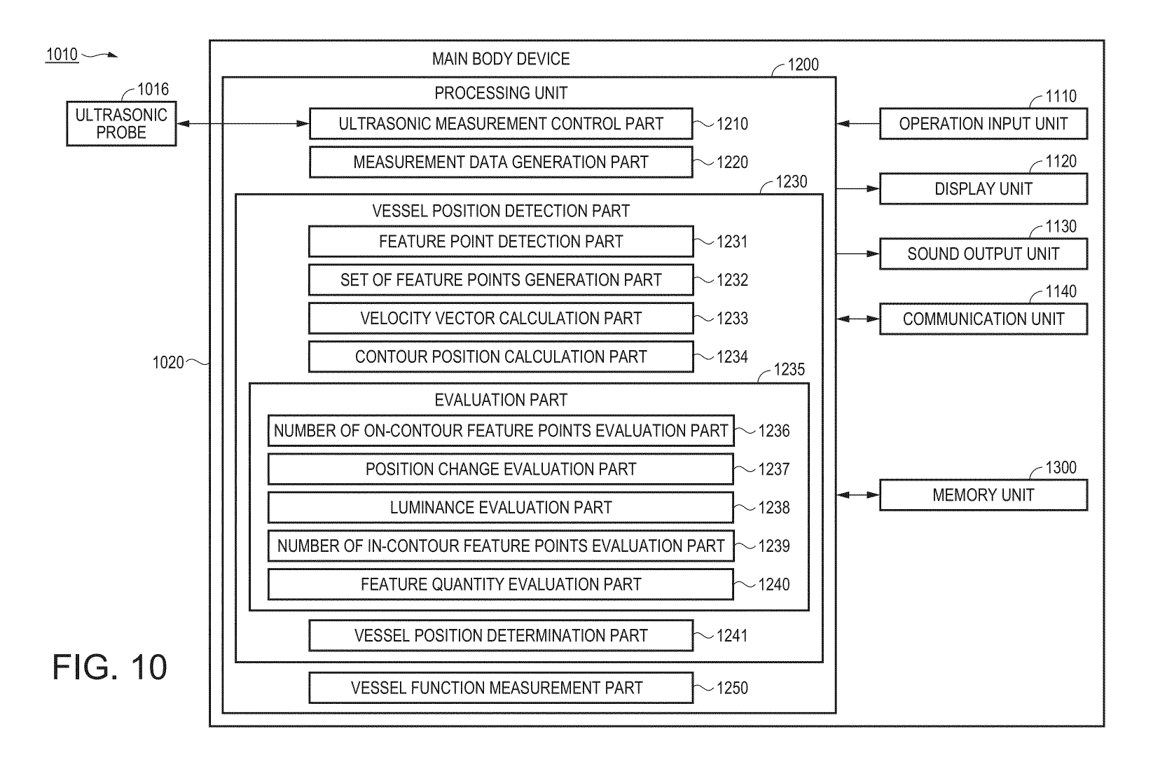

FIG. 10 is a functional configuration diagram of the ultrasonic measuring apparatus 1010 in the first embodiment. As shown in FIGS. 1 and 10, the ultrasonic measuring apparatus 1010 includes the main body device 1020 and the ultrasonic probe 1016. The main body device 1020 includes an operation input unit 1110, a display unit 1120, a sound output unit 1130, a communication unit 1140, a processing unit 1200, and a memory unit 1300.

The operation input unit 1110 is realized by input devices including a button switch, a touch panel, various sensors etc., and outputs an operation signal in response to the performed operation to the processing unit 1200. In FIG. 1, the touch panel 1012 and the keyboard 1014 correspond to the unit.

The display unit 1120 is realized by a display device such as an LCD (Liquid Crystal Display) and performs various kinds of display based on display signals from the processing unit 1200. In FIG. 1, the touch panel 1012 corresponds to the unit.

The sound output unit 1130 is realized by a sound output device such as a speaker and performs various kinds of sound output based on sound signals from the processing unit 1200.

The communication unit 1140 is realized by a wireless communication device such as a wireless LAN (Local Area Network) or Bluetooth (registered trademark) or a communication device such as a modem, a jack of a wire communication cable, or a control circuit, and connects to a given communication line and performs communication with an external device. In FIG. 1, the unit corresponds to the communication IC 1028 mounted on the control board 1022.

The processing unit 1200 is realized by a microprocessor such as a CPU (Central Processing Unit) or GPU (Graphics Processing Unit) or an electronic component such as an ASIC (Application Specific Integrated Circuit) or IC (Integrated Circuit) memory, and executes various kinds of calculation processing based on the programs and data stored in the memory unit 1300, the operation signal from the operation input unit 1110, etc. and controls the operation of the ultrasonic measuring apparatus 1010. Further, the processing unit 1200 has an ultrasonic measurement control part 1210, a measurement data generation part 1220, a vessel position detection part 1230, and a vessel function measurement part 1250.

The ultrasonic measurement control part 1210 controls transmission and reception of ultrasonic wave in the ultrasonic probe 1016. Specifically, the part allows the ultrasonic probe 1016 to transmit ultrasonic wave at transmission times at a predetermined cycle. Further, the part performs amplification of a signal of reflected wave of ultrasonic wave received in the ultrasonic probe 1016 etc.

The measurement data generation part 1220 generates measurement data containing image data of the respective modes of the A-mode, B-mode, and M-mode based on the received signals of the reflected wave by the ultrasonic probe 1016.

The vessel position detection part 1230 has a feature point detection part 1231, a set of feature points generation part 1232, a velocity vector calculation part 1233, a contour position calculation part 1234, an evaluation part 1235, and a vessel position determination part 1241, and performs detection of the vessel position based on the measurement data generated by the measurement data generation part 1220.

The feature point detection part 1231 detects feature points in a B-mode image. In the detection of feature points, pixels that satisfy a predetermined condition are detected as feature points based on the luminance of the pixels, luminance differences between the pixels and the surrounding pixels of the pixels, or the like.

The set of feature points generation part 1232 generates a set of feature points including three feature points selected from the detected feature points.

The velocity vector calculation part 1233 compares temporally adjacent B-mode images, and calculates velocity vectors (magnitudes and directions of velocities) of the respective feature points based on the amounts of movements and frame rates of the feature points.

The contour position calculation part 1234 calculates the definitional equation (1) of the circle passing through the three feature points forming the set of feature points (assumed circle). Specifically, the parameters l, m, n in the definitional equation (1) are obtained, and thereby, the contour position of the circle is calculated.

The evaluation part 1235 has a number of on-contour feature points evaluation part 1236, a position change evaluation part 1237, a luminance evaluation part 1238, a number of in-contour feature points evaluation part 1239, and a feature quantity evaluation part 1240, and performs evaluations on the criterion as to whether or not the position of the assumed circle corresponding to the set of feature points is regarded as the position of the vessel wall. Specifically, as shown in the above formula (2), the item evaluation values fi obtained with respect to each of the plurality of evaluation items are multiplied by the weight coefficient ai and added, and thereby, the comprehensive evaluation value F is calculated.

The number of on-contour feature points evaluation part 1236 performs an evaluation based on “number of feature points located on contour of assumed circle” as the first evaluation item. That is, the number j of the feature points q located on the contour of assumed circle 1050 in the B-mode image is obtained, and the probability density obtained from the probability density function h11(j) based on the number j of feature points is used as the evaluation value f11 of the first evaluation item (see FIG. 4).

The position change evaluation part 1237 performs an evaluation based on “position changes of feature points on contour of assumed circle” as the second evaluation item. That is, averages of the displacement velocities (position changes per unit time) of the respective feature points q on the contour of the assumed circle 1050 in the B-mode image over a predetermined period (equal to or more than one heartbeat of a heartbeat period, about several seconds) are used as displacement velocities vi of the feature points, and an average velocity va as an average of the displacement velocities vi of the respective feature points q is obtained. Then, the probability density obtained from the probability density function h12(va) based on the obtained average velocity va is used as the evaluation value f12 of the second evaluation item (see FIG. 5).

The luminance evaluation part 1238 performs an evaluation based on “luminance of feature points on contour” as the third evaluation item. That is, an average value of luminance L of the respective feature points q on the contour of the assumed circle 1050 in the B-mode image is obtained, and the probability density obtained from the probability density function h13(La) based on the obtained average luminance La is used as the evaluation value f13 of the third evaluation item (see FIG. 6).

The number of in-contour feature points evaluation part 1239 performs an evaluation based on “number of feature points inside contour” as the fourth evaluation item. That is, the number k of the feature points r located inside the assumed circle 1050 in the B-mode image is obtained, and the probability density obtained from the probability density function h14(k) based on the obtained number k of feature points is used as the evaluation value f14 of the fourth evaluation item (see FIG. 8).

The feature quantity evaluation part 1240 performs an evaluation based on “feature quantity of external image of assumed circle” as the fifth evaluation item. That is, the degree of approximation of the images is calculated by comparison of the partial image 1052 around the assumed circle 1050 in the B-mode image with the feature image 1054 prepared in advance, and the calculated degree of approximation is used as the evaluation value f15 of the fifth evaluation item (see FIG. 9).

The vessel position determination part 1241 determines the vessel position using the evaluation result with respect to the set of feature points by the evaluation part 1235. Specifically, existence of the vessel wall in the contour position of the assumed circle by the set of feature points having the maximum comprehensive evaluation value F is determined, the center C and the radius R of the assumed circle are obtained, and the vessel position is decided. In this regard, in order to determine the vessel position with higher accuracy, the feature points on the contour of the assumed circle in the B-mode image may be reselected, the contour position may be recalculated using e.g. the least-square method based on the reselected feature points, and the center C and the radius R may be decided based on the recalculated contour position.

The vessel function measurement part 1250 performs measurements of given vessel function information. Specifically, the part performs measurements of vessel function information of the measurement of the vessel diameter, IMT, etc. of the vessel specified by the detected vessel position, the estimation calculation of blood pressure from vessel diameter fluctuations by tracking the vessel anterior wall and the vessel posterior wall, and the calculation of the pulse rate.

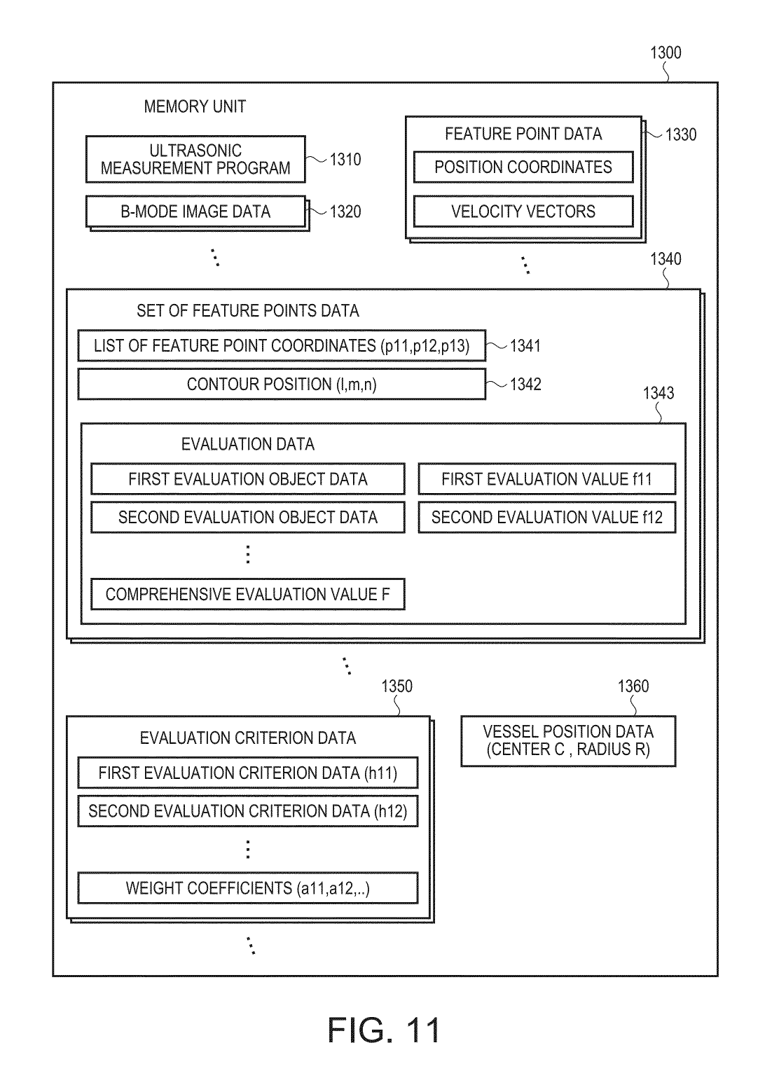

The memory unit 1300 is realized by a memory device such as ROM, RAM, or hard disk, stores programs, data, etc. for integrated control of the ultrasonic measuring apparatus 1010 by the processing unit 1200 and used as a work area of the processing unit 1200, and calculation results executed by the processing unit 1200, operation data from the operation input unit 1110, etc., are temporarily stored therein. In FIG. 1, the part corresponds to the storage medium 1026 mounted on the control board 1022. In the embodiment, as shown in FIG. 11, an ultrasonic measurement program 1310, B-mode image data 1320, feature point data 1330, set of feature points data 1340, evaluation criterion data 1350, and vessel position data 1360 are stored in the memory unit 1300.

The B-mode image data 1320 stores B-mode images generated with respect to each measurement frame associated with frame IDs.

The feature point data 1330 is generated with respect to each detected feature point and stores position coordinates and velocity vectors in the B-mode images in the respective frames.

The set of feature points data 1340 is generated with respect to each set of feature points and stores a list 1341 of the position coordinates of the respective three feature points forming the set of feature points, a contour position 1342 of the assumed circle passing through three the three feature points, and evaluation data 1343 used for evaluations of the feature points. The contour position 1342 stores the parameters l, m, n in the formula (1) defining the assumed circle. The evaluation data 1343 stores evaluation object data and evaluation values for the respective plurality of evaluation items and comprehensive evaluation values.

The evaluation criterion data 1350 stores evaluation criteria (probability density functions h11 to h15, the feature image 1054, etc.) for the respective plurality of evaluation items and the weight coefficients a11 to a15.

The vessel position data 1360 is data of the detected vessel position and stores e.g., the position coordinates of the center C of the short-axis section and the radius R of the vessel.

### Flow of Processing

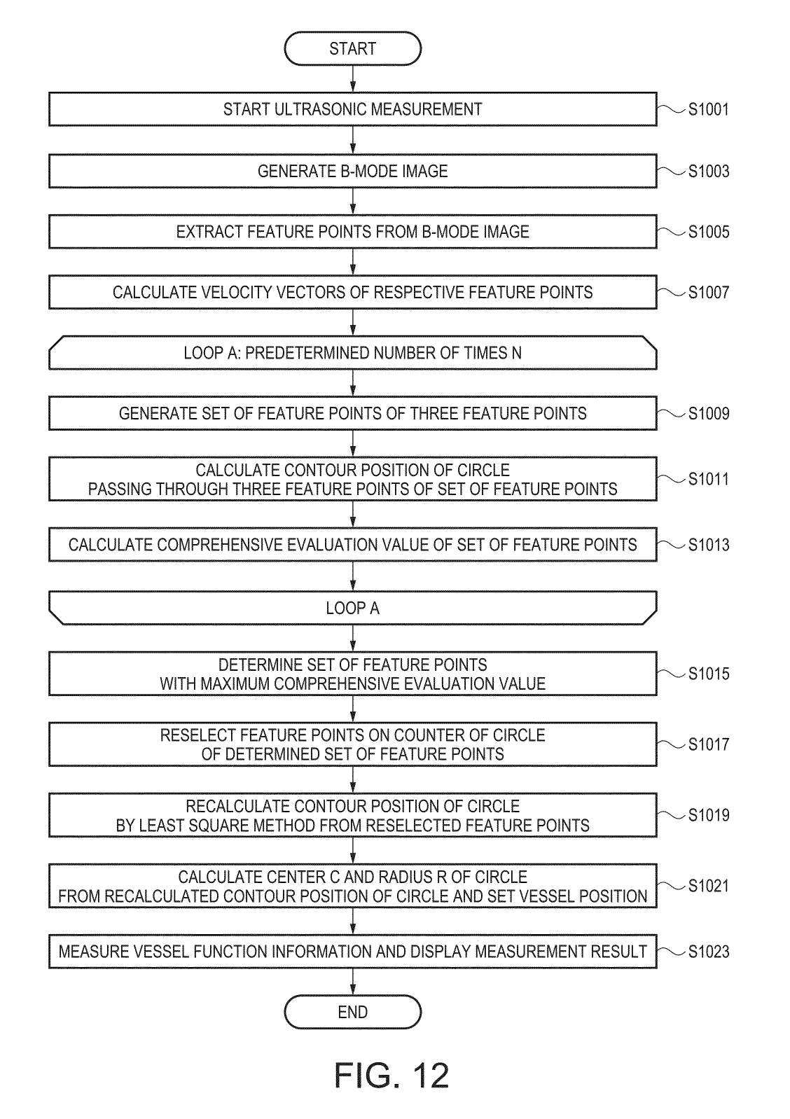

FIG. 12 is a flowchart for explanation of the ultrasonic measurement processing in the first embodiment. The processing is realized by the processing unit 1200 executing the ultrasonic measurement program 1310.

The processing unit 1200 first starts an ultrasonic measurement using the ultrasonic probe 1016 (step S1001). Then, the measurement data generation part 1220 generates a B-mode image based on received signals of ultrasonic reflected wave by the ultrasonic probe 1016 (step S1003). Subsequently, the feature point detection part 1231 detects feature points from the B-mode image (step S1005). Then, the velocity vector calculation part 1233 calculates velocity vectors of the respective detected feature points (step S1007).

Then, the processing of loop A is repeated in a predetermined number of times. In the loop A, the set of feature points generation part 1232 selects three feature points from the feature points detected from the B-mode image and generates a set of feature points of the selected three feature points (step S1009). Then, the contour position calculation part 1234 calculates the parameters of a circle passing through the three feature points forming the generated set of feature points (assumed circle) and calculates a contour position of the circle (step S1011).

Subsequently, the evaluation part 1235 calculates a comprehensive evaluation value F of the set of feature points (step S1013). For calculation of the comprehensive evaluation value F, the number of on-contour feature points evaluation part 1236 obtains the number j of feature points located on the contour of the assumed circle in the B-mode image and probability density obtained from the probability density function h11(j) based on the obtained number j of feature points is used as the evaluation value f11 of the first evaluation item. Further, the position change evaluation part 1237 obtains an average velocity va as an average of displacement velocities Vi of the respective feature points q located on the contour of the assumed circle in the B-mode image and probability density obtained from the probability density function h12(va) based on the obtained average velocity va is used as the evaluation value f12 of the second evaluation item. Furthermore, the luminance evaluation part 1238 obtains an average value of luminance L of the respective feature points q on the contour of the assumed circle 1050 in the B-mode image and probability density obtained from the probability density function h13 (La) based on the obtained average luminance La is used as the evaluation value f13 of the third evaluation item. Further, the number of in-contour feature points evaluation part 1239 obtains the number k of the feature points r located inside the assumed circle 1050 in the B-mode image and probability density obtained from the probability density function h14(k) based on the obtained number k of feature points is used as the evaluation value f14 of the fourth evaluation item. Furthermore, the feature quantity evaluation part 1240 compares a partial image 1052 around the assumed circle 1050 in the B-mode image with a feature image 1054 and calculates a degree of approximation of the images, and the calculated degree of approximation is used as the evaluation value f15 of the fifth evaluation item. Then, the evaluation part 1235 multiplies the calculated evaluation values f11 to f15 of the respective evaluation items by the predetermined weight coefficients a11 to a15 and adds up them, and thereby, calculates a comprehensive evaluation value F. The loop A is performed in the above described manner.

When the processing of the loop A at the predetermined number of times is ended, the vessel position determination part 1241 determines the set of feature points having the maximum comprehensive evaluation value F from all sets of feature points (step S1015). Then, the feature points located on the contour of the assumed circle formed by the determined set of feature points are reselected (step S1017), the parameters of the circle are recalculated by the least-square method using the positions of the reselected feature points, and the contour position of the circle is recalculated (step S1019). Then, the center C and the radius R of the circle are decided from the recalculated contour position and the vessel position is obtained (step S1021).

Then, the vessel function measurement part 1250 performs a measurement of given vessel function information using the transmission and reception results of ultrasonic wave by the ultrasonic probe 1016, and stores and displays the measured vessel (step S1023). This is the end of the ultrasonic measurement processing.

### Advantages

According to the first embodiment, the combination of the feature points in which the positions of the feature points in the ultrasonic image have a location relationship along the section shape of the vessel in the short-axis direction is selected, and the position of the vessel is determined using the evaluation result of the selected combination. In the ultrasonic image containing the section of the vessel in the short-axis direction, there is a characteristic that many feature points appear along the contour of the section shape of the vessel. Thereby, a new technology of detecting the position of the vessel from the location relationship of the positions of the feature points in the ultrasonic image may be realized.

Note that, in the first embodiment, three feature points form the set of feature points, however, four or more feature points may form the set of feature points.

Further, as the evaluation items for evaluation of the set of feature points, the five evaluation items are explained as an example, however, it is not necessary to use all evaluation items for determination of the comprehensive evaluation value F. Of the five evaluation items, one or more selected evaluation items maybe used for determination of the comprehensive evaluation value F. Or, other evaluation items may be used.

### Second Embodiment

Next, the second embodiment will be explained. Note that the second embodiment has some configurations in common with the first embodiment. Accordingly, in the explanation of the second embodiment, the same signs are assigned to the same configurations as those of the first embodiment and their explanation will be omitted or simplified.

### Functional Configuration

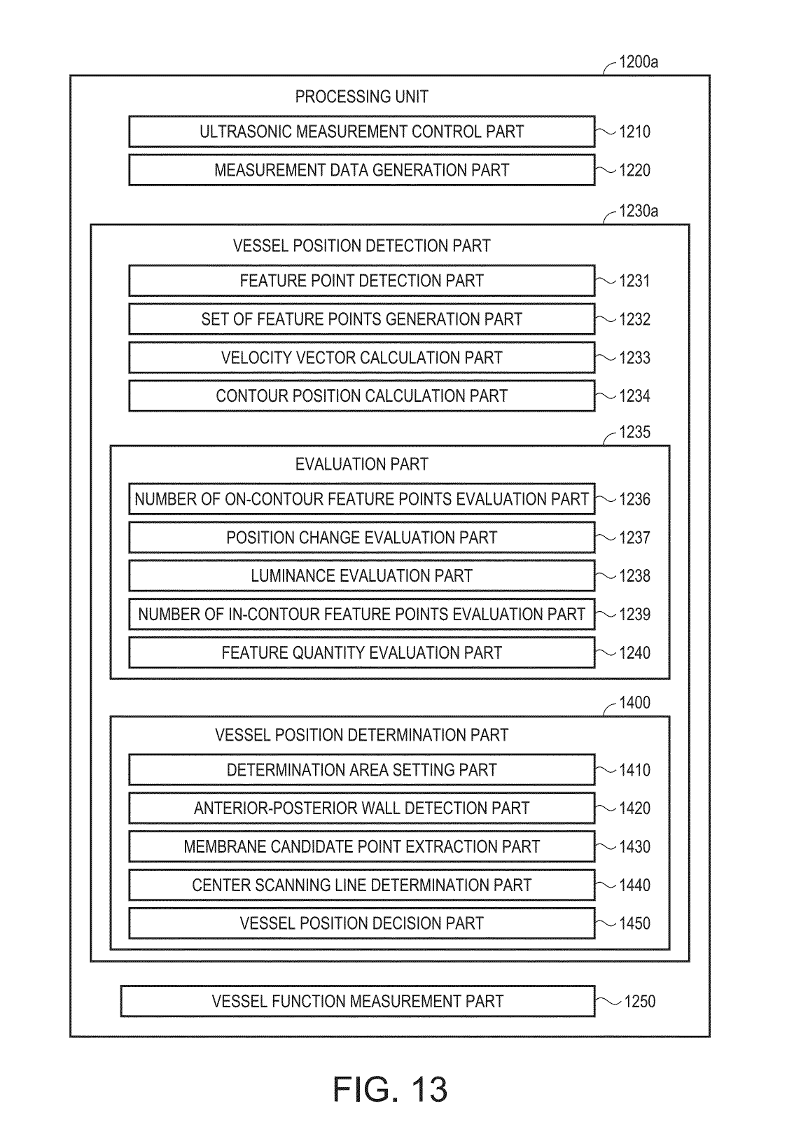

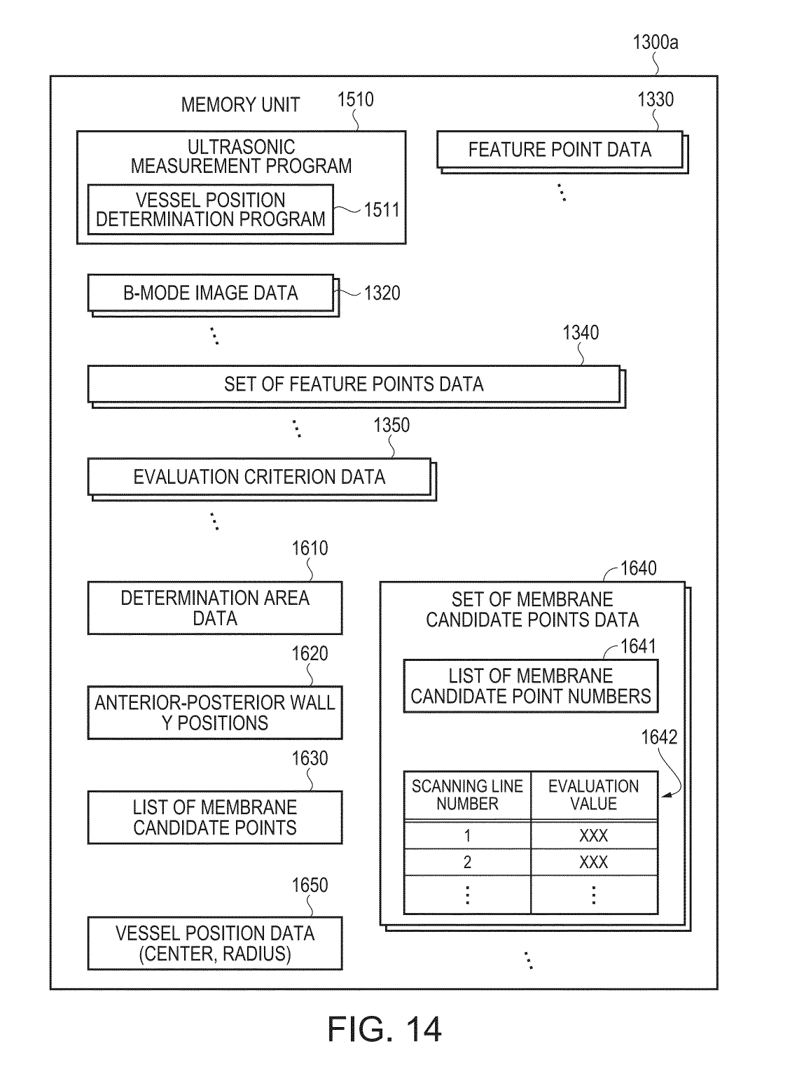

FIG. 13 shows a configuration example of a processing unit 1200a of an ultrasonic measuring apparatus in the second embodiment, and FIG. 14 shows a configuration example of a memory unit 1300a. The ultrasonic measuring apparatus of the second embodiment may be realized by replacing the processing unit 1200 by the processing unit 1200a in FIG. 13 and replacing the memory unit 1300 by the memory unit 1300a in FIG. 14 in the ultrasonic measuring apparatus 1010 of the first embodiment shown in FIG. 10.

As shown in FIG. 13, the processing unit 1200a has the ultrasonic measurement control part 1210, the measurement data generation part 1220, a vessel position detection part 1230a, and the vessel function measurement part 1250. Further, in the vessel position detection part 1230a, a vessel position determination part 1400 has a determination area setting part 1410, an anterior-posterior wall detection part 1420, a membrane candidate point extraction part 1430, a center scanning line determination part 1440, and a vessel position decision part 1450.

The determination area setting part 1410 obtains the contour position of the vessel in the B-mode image using the evaluation results by the evaluation part 1235, and sets a determination area of the vessel position based on the obtained contour position.

The anterior-posterior wall detection part 1420 detects positions of the anterior wall and the posterior wall of the vessel in the Y direction (depth direction from the living body surface) in the determination area.

The membrane candidate point extraction part 1430 extracts membrane candidate points of the adventitia (anterior-wall adventitia candidate point and posterior-wall adventitia candidate point) and membrane candidate points of the lumen-intima boundaries (anterior wall intima candidate point and posterior wall intima candidate point) as respective feature points based on the Y positions of the anterior wall and the posterior wall.

The center scanning line determination part 1440 uses a combination of the membrane candidate points, and determines a scanning line passing through the center of the vessel (hereinafter, referred to as “center scanning line”) of a plurality of scanning lines with respect to the transmission and reception of the ultrasonic probe 1016. The center scanning line determination part 1440 evaluates luminance of the membrane candidate points contained in sets of membrane candidate points with respect to each set of membrane candidate points as a combination of the membrane candidate points using a predetermined evaluation calculation. Then, the scanning line with respect to the set of membrane candidate points receiving the highest evaluation (hereinafter, referred to as “most-highly-evaluated set of membrane candidate points” is specified as the center scanning line.

Here, the scanning lines correspond to the respective rows of pixels in the Y-direction in the B-mode image (in the embodiment, the determination area set in the B-mode image), and are identified by scanning line numbers assigned to the respective positions of the determination area in the X direction.

The vessel position decision part 1450 decides the center and the radius (or diameter) of the vessel as the vessel position using the most-highly-evaluated set of membrane candidate points according to the center scanning line.

In the memory unit 1300a, an ultrasonic measurement program 1510, the B-mode image data 1320, the feature point data 1330, the set of feature points data 1340, the evaluation criterion data 1350, determination area data 1610, an anterior-posterior wall Y position 1620, a list of membrane candidate points 1630, set of membrane candidate points data 1640, and vessel position data 1650 are stored.

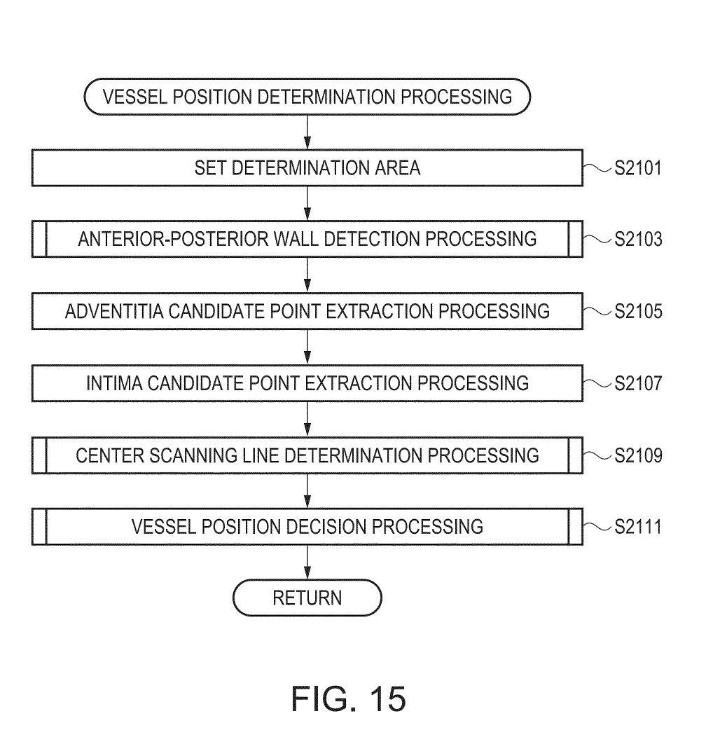

The ultrasonic measurement program 1510 contains a vessel position determination program 1511 for execution of vessel position determination processing (see FIG. 15).

The determination area data 1610 stores a set range of the determination area set in the B-mode image. The anterior-posterior wall Y position 1620 stores the Y positions of the anterior wall and the posterior wall detected in the determination area. The list of membrane candidate points 1630 stores position coordinates (X,Y) of the respective membrane candidate points extracted in the determination area. The set of membrane candidate points data 1640 is generated with respect to each membrane candidate point and stores a list 1641 of membrane candidate point numbers assigned to the membrane candidate points contained in the set of membrane candidate points and evaluation values 1642 with respect to each scanning line for the set of membrane candidate points.

### Flow of Processing

FIG. 15 is a flowchart showing a flow of vessel position determination processing in the second embodiment. In the second embodiment, in the ultrasonic measurement processing of the first embodiment shown in FIG. 12, the vessel position determination part 1400 performs vessel position determination processing shown in FIG. 15 in place of the processing at step S1021. The processing is realized by the vessel position determination part 1400 executing the vessel position determination program 1511.

First, the determination area setting part 1410 sets a determination area having a strip shape along the Y direction in the B-mode image to contain the center of the contour position obtained at step S1019 in FIG. 12 at the upstream (step S2101).

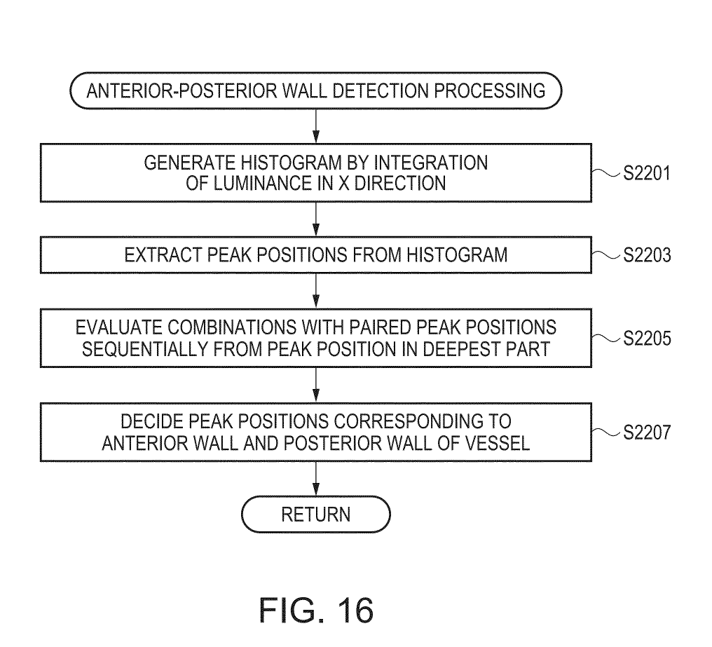

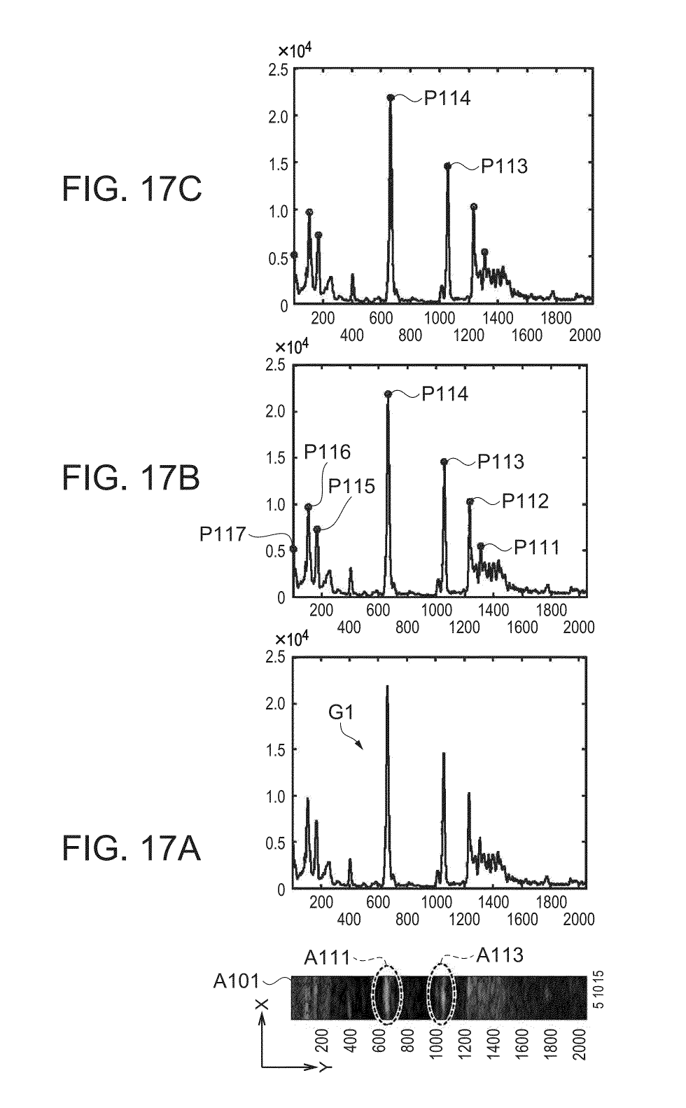

Subsequently, the anterior-posterior wall detection part 1420 detects the Y positions of the anterior wall and the posterior wall of the vessel in the determination area using e.g., B-mode image data of the determination area set at step S2101 (step S2103: anterior-posterior wall detection processing). FIG. 16 is a flowchart showing a flow of the anterior-posterior wall detection processing in the second embodiment. Further, FIGS. 17A to 17C are diagrams for explanation of the anterior-posterior wall detection processing in the second embodiment.

The anterior-posterior wall detection part 1420 first generates a histogram by integration in the X direction (along the living body surface) of luminance of the determination area in the respective positions in the Y direction (step S2201). The right part of FIG. 17A shows an example of a histogram G1 with a B-mode image of a determination area A101 on the side as the left part. The upper side of FIG. 17A is the surface layer side (the living body surface side in contact with the ultrasonic probe 1016), and an anterior wall part A111 and a posterior wall part A113 of the vessel in the determination area A101 are shown by surrounding broken lines. Here, the width in the X direction of the determination area A101 set in the upstream processing (step S101 in FIG. 15) may be appropriately set. In FIG. 17A, the number of pixels (number of scanning lines) in the X direction is shown as “15”, and the determination area A101 in FIG. 17A includes 15 scanning lines with scanning line numbers of “1” to “15”. As shown in the histogram G1 of the determination area A101, the luminance is integrated in the X direction, and thereby, the integrated value becomes larger in the Y positions of the anterior wall A111 and the posterior wall A113 in which the reflectance of ultrasonic wave is higher and the luminance is higher.

Then, the anterior-posterior wall detection part 1420 searches for peak values of the integrated values from the histogram generated at step S2201, and extracts the Y positions thereof as peak positions (step S2203). FIG. 17B shows a plurality of peak positions P111 to P117 extracted from the histogram in FIG. 17A. For the processing here, e.g., a method of extracting Y positions in which changes of the integrated values show convex shapes based on magnitude relationships with the integrated values in the Y positions before and after may be used. Specifically, the Y positions having the integrated values larger than the integrated values in the Y positions immediately before and having the integrated values larger than the integrated values in the Y positions immediately after are extracted as the peak positions. Or, a method of extracting the Y positions in which positive and negative signs change as a result of first derivation as the peak positions may be used.

Then, the anterior-posterior wall detection part 1420 makes combinations of two of the peak positions extracted at step S2203, and evaluates appropriateness of the combined two peak positions as the anterior wall and the posterior wall of the vessel (step S2205l). The combinations are created by respectively paring the different peak positions sequentially from the peak position in the deepest part (sequentially from the peak position P111 in the example of FIG. 17B). The evaluation is performed from the deepest part in order to avoid false detection of the peak positions as the anterior and posterior walls because muscle fibers, surrounding tissues, etc. exist at the surface layer side and the luminance is larger due to the existence. Thereby, false detection of the anterior and posterior walls of the vessel may be prevented.

Then, the anterior-posterior wall detection part 1420 checks the distances between the combined two peak positions against the average diameter value of the vessel as the measuring object (the average diameter value of the carotid artery in the embodiment), and evaluates whether or not the respective peak positions of the combinations are appropriate as the anterior wall and the posterior wall of the vessel. When the distances between the peak positions are largely different from the average diameter value used for the checking, evaluations that they are not the combinations corresponding to the anterior wall and the posterior wall may be made. In addition, the anterior-posterior wall detection part 1420 performs evaluations according to whether or not there is another peak position between the combined two peak positions. Blood flows between the anterior wall and the posterior wall, and amplitudes with higher luminance are harder to be generated. Therefore, an evaluation that the combination with another peak existing in between does not correspond to the anterior wall and the posterior wall may be made. If the evaluation that the combined two peak positions do not correspond to the anterior wall and the posterior wall is made, the processing moves to an evaluation of the next combination.

Then, the anterior-posterior wall detection part 1420 performs evaluations of the combinations of the two peak positions sequentially from the deepest part as described above, and thereby, decides the peak positions corresponding to the anterior wall and the posterior wall (step S2207). For example, as the example in FIG. 17C, if the combinations of the two peak positions P113, P114 among the peak positions P111 to P117 shown in FIG. 17B are evaluated to correspond to the anterior wall and the posterior wall, the peak position P114 is decided and detected as the Y position of the anterior wall and the peak position P113 is decided and detected as the Y position of the posterior wall.

Note that processing of rearranging the respective peak positions extracted at step S2203 prior to the evaluations at step S2205 in the descending order of the integrated values may be performed and the appropriateness evaluations of the anterior and posterior walls by combining two peak positions sequentially from the peak positions having the larger integrated values may be performed. This is because the peak positions having the larger integrated values have higher probabilities of corresponding to the anterior wall and the posterior wall of the vessel.

Returning to FIG. 15, subsequently, the membrane candidate point extraction part 1430 performs adventitia candidate point extraction processing and extracts anterior-wall adventitia candidate points and the posterior-wall adventitia candidate points (step S2105). Further, the membrane candidate point extraction part 1430 performs intima candidate point extraction processing and extracts anterior-wall intima candidate points and the posterior-wall intima candidate points (step S2107).

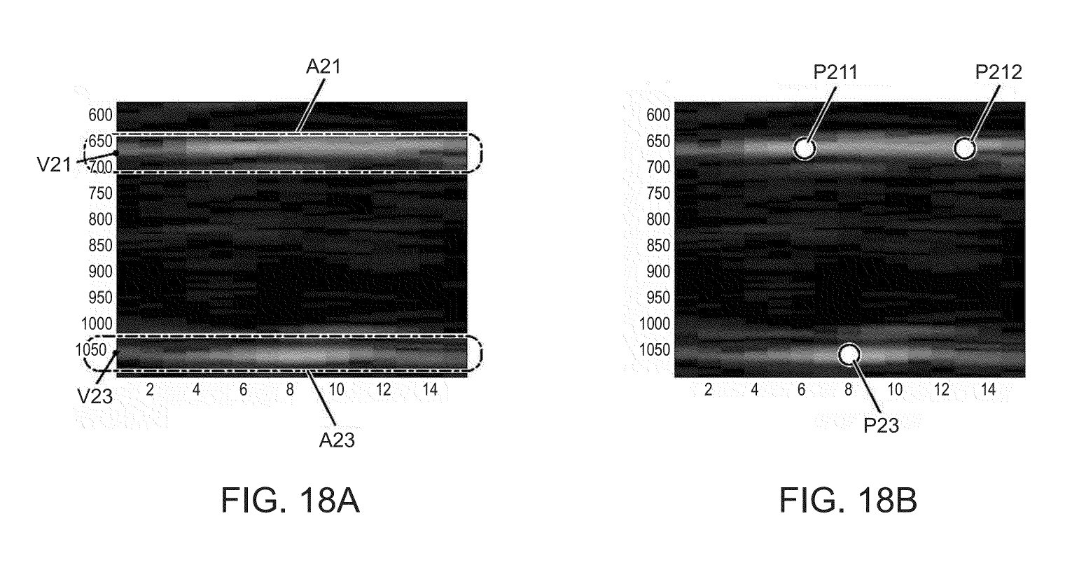

FIGS. 18A and 18B are diagrams for explanation of the adventitia candidate point extraction processing in the second embodiment. In the adventitia candidate point extraction processing, the membrane candidate point extraction part 1430 first sets adventitia search areas in the determination area based on the Y positions of the anterior wall and the posterior wall detected by the anterior-posterior wall detection processing in FIG. 16. For example, as shown by surrounding dashed-dotted lines in FIG. 18A, areas having predetermined depth ranges respectively around a Y position V21 of the anterior wall and a Y position V23 of the posterior wall are set as adventitia search areas A21, A23. The widths of the adventitia search areas A21, A23 in the Y direction are determined in advance in consideration of the amounts of dilatation and constriction of the vessel and the amounts of relative movements of the vessel position due to beats. Then, the membrane candidate point extraction part 1430 extracts luminance peak positions from the respective adventitia search areas A21, A23 using B-mode image data of the adventitia search areas A21, A23. For the processing here, e.g., a method of extending the search in the Y direction explained at step S2203 in FIG. 16 to two dimensions may be used, and the respective adventitia search areas A21, A23 are searched for luminance in the Y direction and X direction and a plurality of peak positions in which the luminance is the locally maximum from all of the adventitia search areas A21, A23. Then, as shown by white circles in the example of FIG. 18B, the membrane candidate point extraction part 1430 sets peak positions P211, P212 extracted from the anterior-wall adventitia search area A21 as anterior-wall adventitia candidate points and a peak position P23 extracted from the posterior-wall adventitia search area A23 as a posterior-wall adventitia candidate point.

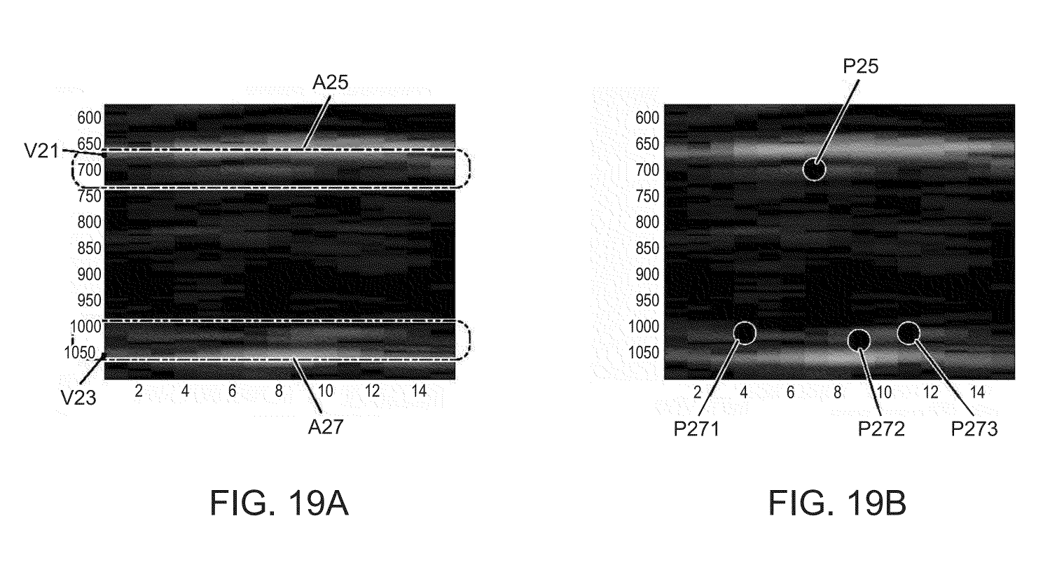

Further, FIGS. 19A and 19B are diagrams for explanation of the intima candidate point extraction processing in the second embodiment. The intima candidate point extraction processing may be performed in the same procedure as that of the adventitia candidate point extraction processing, however, for setting of the intima search area, a condition that the lumen-intima boundaries exist closer to the lumen side of the vessel than the adventitia is considered. Specifically, as shown by surrounding dashed-two dotted lines in FIG. 19A, an area having a predetermined depth range around a Y position apart at a predetermined distance to the depth side from the Y position V21 of the anterior wall is set as an intima search area A25 and an area having a predetermined depth range around a Y position apart at a predetermined distance to the surface layer side from the Y position V23 of the posterior wall is set as an intima search area A27. The predetermined distances are determined in consideration of the standard IMT (wall thickness) length in advance. The widths of the intima search areas A25, A27 in the Y direction are determined in advance like the adventitia search areas A21, A23. Then, a plurality of peak positions in which the luminance is the locally maximum from all of the intima search areas A25, A27 in the same manner as that of the adventitia candidate point extraction processing. Then, as shown by black circles in the example of FIG. 19B, the membrane candidate point extraction part 1430 sets a peak position P25 extracted from the anterior-wall intima search area A25 as an anterior-wall adventitia candidate point and peak positions P271 to P273 extracted from the posterior-wall intima search area A27 as posterior-wall adventitia candidate points.

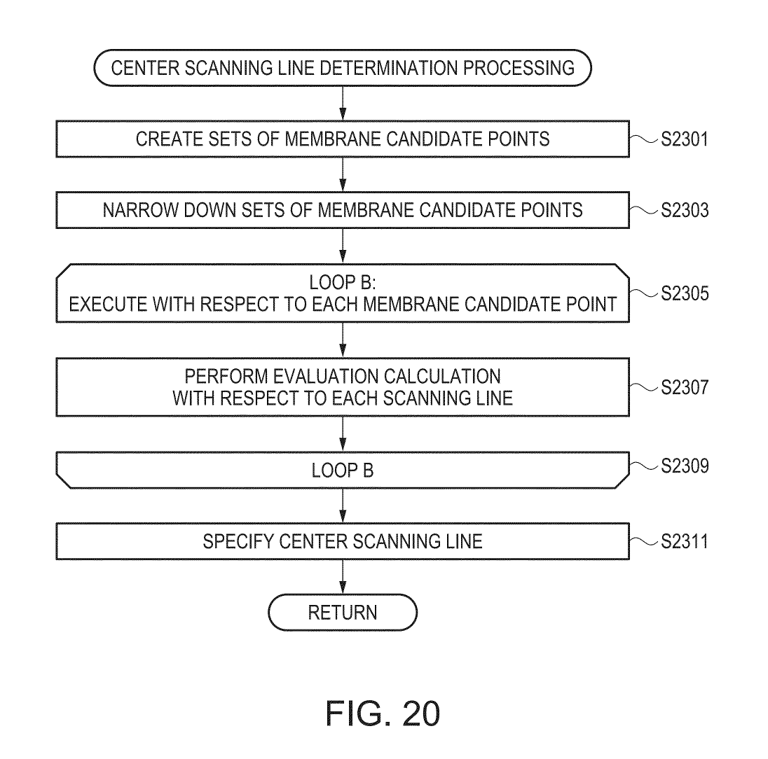

Returning to FIG. 15, subsequently, the center scanning line determination part 1440 performs center scanning line determination processing and determines a center scanning line (step S2109). FIG. 20 is a flowchart showing a flow of the center scanning line determination processing in the second embodiment.

In the center scanning line determination processing, the center scanning line determination part 1440 first creates sets of membrane candidate points by combining a plurality of membrane candidate points extracted at steps S2105, S2107 in FIG. 15 (step S2301). For example, the part creates all combinations of four membrane candidate points by combining each one of the anterior-wall adventitia candidate points, the posterior-wall adventitia candidate points, the anterior-wall intima candidate points, and the posterior-wall intima candidate points, and uses the respective sets as the sets of membrane candidate points. Note that the sets of membrane candidate points may be combinations of two or more of the membrane candidate points, and, for example, all combinations of the two or more membrane candidate points may be created and the respective sets may be used as the sets of membrane candidate points. Or, a predetermined number of sets of membrane candidate points maybe selected from the created sets of membrane candidate points.

Then, the center scanning line determination part 1440 evaluates appropriateness of the respective membrane candidate points contained in the sets of membrane candidate points as the respective positions of the anterior-wall adventitia, the posterior-wall adventitia, the anterior-wall lumen-intima boundary, or the posterior-wall lumen-intima boundary with respect to the sets of membrane candidate points created at step S2301, and narrows down the sets of membrane candidate points as processing objects in downstream loop B (step S2303). For example, the center scanning line determination part 1440 checks the distances between the anterior-wall adventitia candidate points and the posterior-wall adventitia candidate points against the average diameter value of the vessel as the measuring object, and excludes the set of membrane candidate points largely different from the average diameter value from the processing objects. A configuration of checking the distances between the anterior-wall adventitia candidate points and the posterior-wall adventitia candidate points against the average diameter value and narrowing down the sets may be employed. Further, the part respectively checks the distances between the anterior-wall adventitia candidate points and the anterior-wall intima candidate points and the distances between the posterior-wall adventitia candidate points and the posterior-wall intima candidate points against the IMT length, and excludes the set of membrane candidate points having one or both of the distances not nearly equal to the IMT length from the processing objects. Furthermore, the part extrudes, of the membrane candidate points contained in the sets of membrane candidate points, the sets of membrane candidate points having distances in the X direction between the two membrane candidate points farthest from each other in the X direction from the processing objects.

Then, the center scanning line determination part 1440 sequentially sets the sets of membrane candidate points not extruded at step S2302, but left as processing objects, and performs processing of loop B (steps S2305 to S2309).

That is, in the loop B, the center scanning line determination part 1440 performs predetermined evaluation calculations with respect to each scanning line based on the B-mode image data of the determination areas using the sets of membrane candidate points as the processing objects (step S2307). The evaluation calculations are performed by sequentially providing scanning numbers of “1” to “15” to the formula (3) and calculating evaluation values Eval with respect to each scanning line number. In the following formula (3), n represents the total number of sets of membrane candidate points, LineNum represents the scanning line number, (Xₐₙₜₑᵣᵢₒᵣ, Yₐₙₜₑᵣᵢₒᵣ) represents position coordinates of the anterior-wall adventitia candidate point, (Xₚₒₛₜₑᵣᵢₒᵣ, Yₚₒₛₜₑᵣᵢₒᵣ) represents position coordinates of the posterior-wall adventitia candidate point, (xₐₙₜₑᵣᵢₒᵣ, Yₐₙₜₑᵣᵢₒᵣ) represents position coordinates of the anterior-wall intima candidate point, (xₚₒₛₜₑᵣᵢₒᵣ, yₚₒₛₜₑᵣᵢₒᵣ) represents position coordinates of the posterior-wall intima candidate point, respectively. AMP refers to luminance in the position coordinates.

$${Eval}_{n\_LineNum} = \frac{AMP ( LineNum , {Y}_{anterior} )}{AMP ( {X}_{anterior} , {Y}_{anterior} )} + \frac{AMP ( LineNum , {Y}_{posterior} )}{AMP ( {X}_{posterior} , {Y}_{posterior} )} + \frac{AMP ( LineNum , {y}_{anterior} )}{AMP ( {x}_{anterior} , {y}_{anterior} )} + \frac{AMP ( LineNum , {y}_{posterior} )}{AMP ( {x}_{posterior} , {y}_{posterior} )} \qquad ( 3 )$$

Then, after the evaluation calculations at step S2307 with all sets of membrane candidate points as the processing objects, the center scanning line determination part 1440 specifies the most-highly-evaluated scanning line having the largest evaluation value as the center scanning line, and sets the set of membrane candidate points used for the evaluation as the most-highly-evaluated set of membrane candidate points (step S2311).

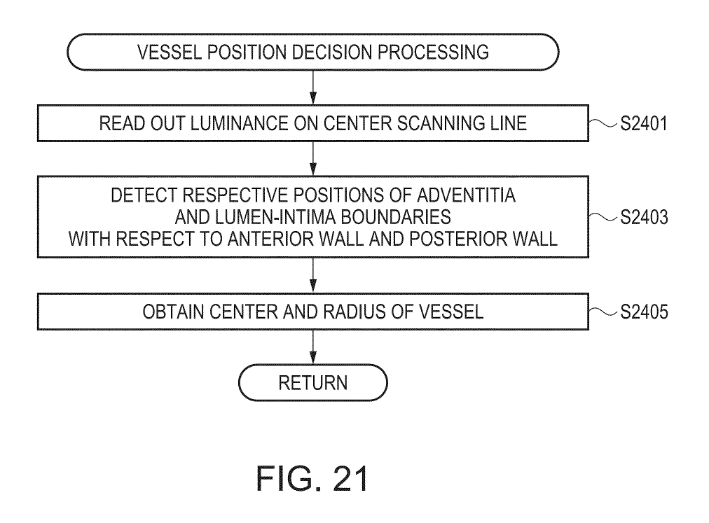

Returning to FIG. 15, subsequently, the vessel position decision part 1450 performs vessel position decision processing and determines the position of the vessel (step S2111). FIG. 21 is a flowchart showing a flow of the vessel position decision processing in the second embodiment.

The vessel position decision part 1450 first refers to the B-mode image data 1320 and reads out luminance of one row on the center scanning line (step S2401). Then, the vessel position decision part 1450 detects the respective positions of the anterior-wall adventitia, the posterior-wall adventitia, the anterior-wall lumen-intima boundary, and the posterior-wall lumen-intima boundary (step S2403). Specifically, first, the vessel position decision part 1450 searches the luminance on the center scanning line read out at step S2401 for peak values, and extracts peak positions. The processing here maybe performed in the same manner as that at step S2203 in FIG. 16. Then, the vessel position decision part 1450 selects each one peak position near the respective Y positions based on the Y positions of the respective membrane candidate points contained in the most-highly-evaluated set of membrane candidate points so that the peak position most closest to the anterior-wall adventitia candidate point in Y position maybe set as the anterior-wall adventitia position.

Note that, at step S2401, luminance of three rows of the respective scanning lines of the center scanning line and both adjacent lines may be read out and integrated values obtained by integration of the luminance of the three rows in the respective positions in the Y direction maybe calculated. Then, the processing at step S2403 may be performed using the integrated values, and the anterior-wall adventitia position, the posterior-wall adventitia position, the anterior-wall lumen-intima boundary position, and the posterior-wall lumen-intima boundary position may be detected from the peak positions of the integrated values. Thereby, the effect of noise may be reduced.

Then, the vessel position decision part 1450 obtains an intermediate position between the anterior-wall adventitia position and the posterior-wall adventitia position detected at step S2403 as the center of the vessel, and obtains the radius of the vessel using the distance between the anterior-wall lumen-intima boundary position and the posterior-wall lumen-intima boundary position as the vessel diameter (step S2405). Note that the radius of the vessel may be obtained using the distance between the anterior-wall adventitia position and the posterior-wall adventitia position as the vessel diameter. Or, not the radius, but the diameter may be obtained.

Then, the processing moves to step S1023 in FIG. 12, and the vessel function measurement part 1250 performs measurements of vessel function information.

In the B-mode image, all areas of the vessel walls do not necessarily clearly appear, and it is possible that the adventitia positions and the lumen-intima boundary positions according to the respective anterior wall and posterior wall can not be properly detected. On the other hand, when the peak positions of luminance are extracted in the B-mode image, other locations with higher luminance due to existence of surrounding tissues, the effect of noise, or the like maybe extracted than the adventitia positions and the lumen-intima boundary positions. In contrast, according to the second embodiment, using the membrane candidate points detected by extraction of the peak positions of luminance in the B-mode image in combination, the respective scanning lines may be evaluated with respect to each set of membrane candidate points in consideration of the relative position relationships of the membrane candidate points contained in the sets of membrane candidate points and the magnitude relationships of luminance at the respective membrane candidate points. Then, the scanning line related to the sets of membrane candidate points receiving the highest evaluation (most-highly-evaluated set of membrane candidate points) may be specified as the center scanning line, and thereby, the position of the vessel on the center scanning line may be determined with higher accuracy using the most-highly-evaluated set of membrane candidate points.

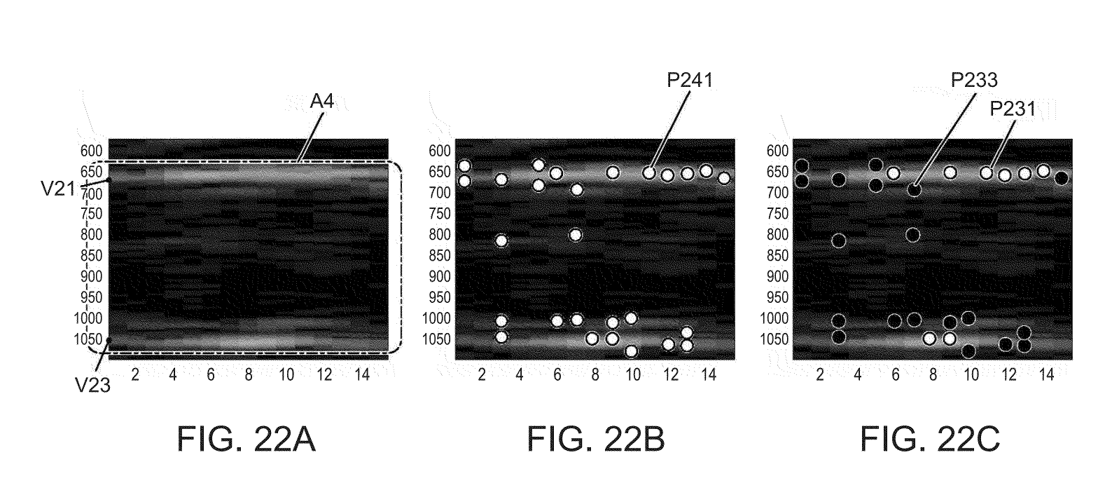

Note that the procedures of the adventitia candidate point extraction processing and the intima candidate point extraction processing are not limited to the methods explained with reference to FIGS. 18A, 18B, 19A, 19B. FIGS. 22A to 22C are diagrams for explanation of a modified example of the adventitia candidate point extraction processing and the intima candidate point extraction processing. In the modified example, the adventitia candidate points and the intima candidate points are detected at the same time. First, as shown in FIG. 22A, one search area A4 is set to contain all ranges of the area of the vessel in the determination area based on the Y position V21 of the anterior wall and the Y position V23 of the posterior wall detected by the anterior-posterior wall detection processing in FIG. 16. The setting of the search area A4 is made in consideration of the above described amounts of dilatation and constriction of the vessel, amounts of relative movements of the vessel position due to beats, etc.

Then, as shown in FIG. 22B, a plurality of feature points P241 in the search area A4 are extracted using the B-mode image data in the search area A4. As the method of extracting the feature points, e.g., a corner detection method (Harris and Stephens) may be used. Or, another corner detection method such as an eigenvalue method (Shi and Tomasi) or FAST feature detection may be used, or feature points may be extracted using local feature quantities represented by SIFT (Scale invariant feature transform) or SURF (Speeded Up Robust Features) feature quantities. Further, a method of performing the search in the depth direction explained at step S2203 in FIG. 16 with respect to all scanning lines (X positions) and extracting peak positions of luminance as feature points may be used.

Then, the extracted feature points P241 are classified into two groups of a group of adventitia candidate points P231 shown by white circles in FIG. 22C and a group of intima candidate points P233 shown by black circles using their luminance. For the classification, a technique of clustering such as the k-means method may be used. The higher luminance of the adventitia part than the luminance of the lumen-intima boundary part enables the grouping. Note that grouping may be performed using gradients of luminance at the respective feature points.

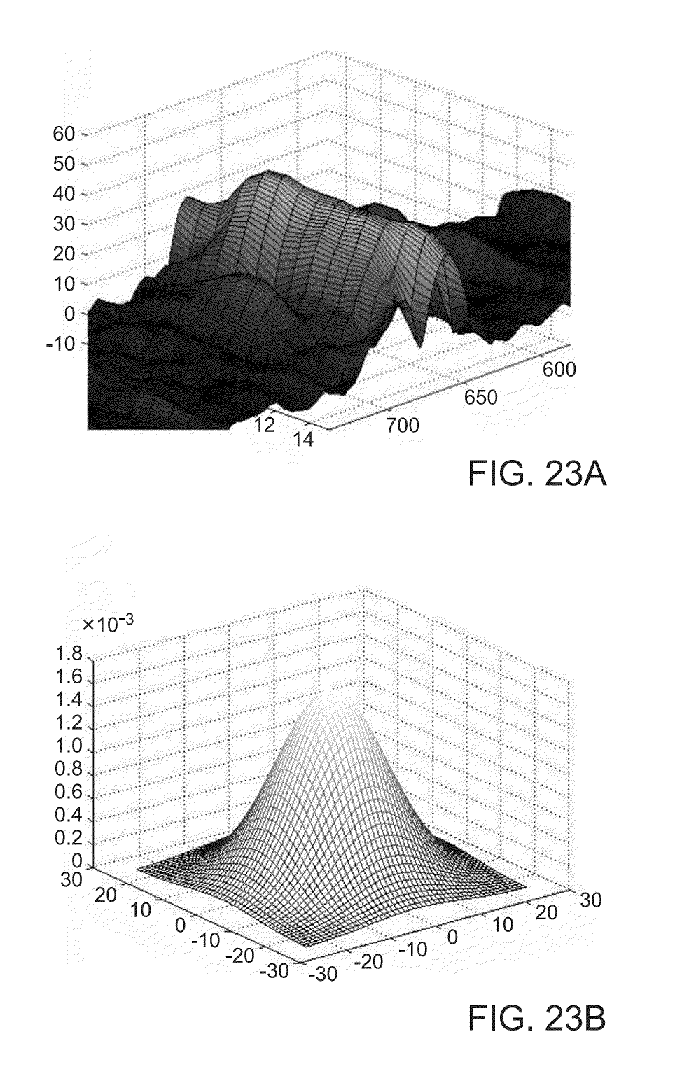

Further, when the membrane candidate points are extracted by the method of the modified example, the center scanning line may be determined by performing the following processing in place of the processing at step S2307 in FIG. 20. That is, first, the respective membrane candidate points contained in the sets of membrane candidate points as processing objects are modeled in a two-dimensional normal distribution using luminance of the surrounding areas of the membrane candidate points. FIG. 23A shows an example of a distribution of luminance of the anterior wall part, and FIG. 23B shows a two-dimensional normal distribution model obtained by modeling a luminance distribution of a surrounding area of a certain membrane candidate point. For example, a two-dimensional normal distribution model representing each membrane candidate point by position coordinates (X,Y) of the membrane candidate point, a breadth (σₓ,σ_{y}) of the luminance distribution with the membrane candidate point as an apex, an amplitude value (amp) representing the height of the apex, etc. is created.

Then, the center scanning line is determined with the two-dimensional normal distribution models (X, Y, σₓ, σ_{y}, amp) of the respective membrane candidate points as input using a statistical model such as a machine learning model including a neural network and a support vector machine (SVM) after previous learning. As a result of the determination with respect to each set of membrane candidate point, the scanning line that was most frequently determined as the center scanning line is set as the center scanning line and the set of membrane candidate points used for the determination is set as the most-highly-evaluated set of membrane candidate points. Note that, the technique of the modified example is preferably applied to the case where the processing performance of the main body device 1020 is higher because the amount of calculation is larger than that of the technique in the above described second embodiment.

According to the modified example, the respective scanning lines may be evaluated with respect to each set of membrane candidate points in consideration of the luminance distributions of the surrounding areas with the apexes as the respective membrane candidate points in addition to the relative position relationships of the membrane candidate points contained in the sets of membrane candidate points and the magnitude relationships of luminance at the respective membrane candidate points. Therefore, the vessel position may be determined with higher accuracy.

Further, the processing explained to use the B-mode image data in the above described vessel position determination processing may be performed using A-mode data (amplitude values) or RF signals in place of the B-mode image data.

Furthermore, in the second embodiment, first, the contour position of the vessel section in the B-mode image is obtained by the method of the first embodiment and the determination area is set to contain the center of the obtained contour position, however, it is not necessary to obtain the contour position by the method of the first embodiment as long as the strip-shaped determination area containing the center of the vessel in the B-mode image may be set. In addition, if the ultrasonic probe 1016 may be positioned immediately above the center of the vessel and a strip-shaped B-mode image containing the center of the vessel may be generated in single ultrasonic measurement performed at step S1001 in FIG. 12, the processing at the step S1203 and the subsequent steps in FIG. 15 may be performed after step S1001 in FIG. 12 without the processing of obtaining the contour position.

### Advantages

According to the second embodiment, the combination of the feature points in which the positions of the feature points in the ultrasonic image have a location relationship along the section shape of the vessel in the short-axis direction is selected, and the position of the vessel is determined using the evaluation result of the selected combination. In the ultrasonic image containing the section of the vessel in the short-axis direction, there is a characteristic that many feature points appear along the contour of the section shape of the vessel. Thereby, a new technology of detecting the position of the vessel from the location relationship of the positions of the feature points in the ultrasonic image may be realized.

Note that, in the second embodiment, three feature points form the set of feature points, however, four or more feature points may form the set of feature points.

Further, as the evaluation items for evaluation of the set of feature points, the five evaluation items are explained as an example, however, it is not necessary to use all evaluation items for determination of the comprehensive evaluation value F. Of the five evaluation items, one or more selected evaluation items maybe used for determination of the comprehensive evaluation value F. Or, other evaluation items may be used.

### Third Embodiment

Next, the third embodiment of the invention will be explained.

### System Configuration

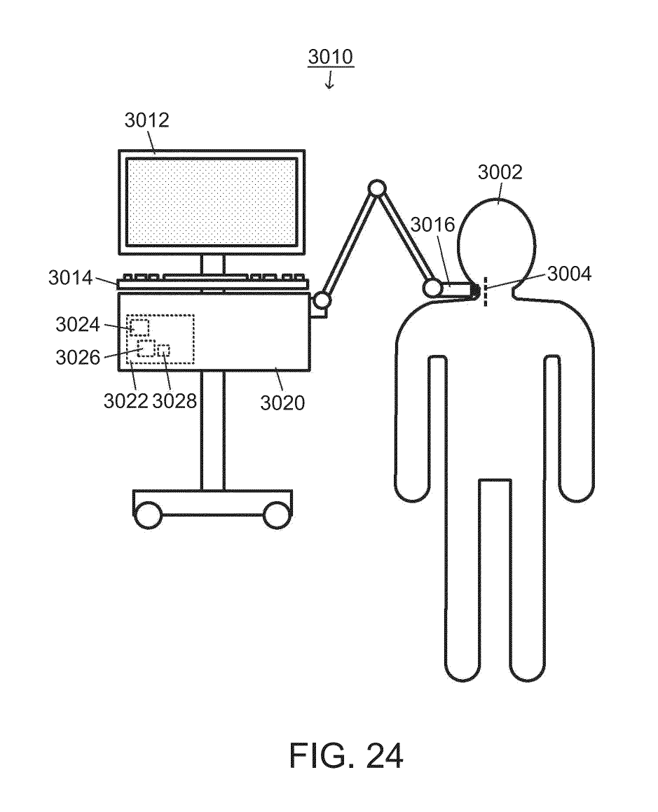

FIG. 24 shows a configuration example of an ultrasonic measuring apparatus 3010 in the third embodiment. The ultrasonic measuring apparatus 3010 is an apparatus that measures biological information of a subject using ultrasonic wave. In the embodiment, a vessel as a measuring object is a carotid artery and, as biological information, vessel function information such as IMT (Intima Media Thickness) is measured. Obviously, other vessel function information such as measurements of a pulse wave propagation velocity and a hardness index value of the vessel wall and a measurement of vessel diameter and a measurement of blood pressure from the vessel diameter may be performed. Further, the vessel as the measuring object may be another artery such as a radial artery.

The ultrasonic measuring apparatus 3010 includes a touch panel 3012, a keyboard 3014, an ultrasonic probe 3016, and a main body device 3020. A control board 3022 is mounted on the main body device 3020 and connected to the respective parts of the touch panel 3012, the keyboard 3014, the ultrasonic probe 3016, etc. so that signals can be transmitted and received.

On the control board 3022, a CPU (Central Processing Unit) 3024, an ASIC (Application Specific Integrated Circuit), various integrated circuits, a storage medium 3026 of an IC memory or a hard disk, and a communication IC 3028 that realizes data communication with an external device are mounted. The main body device 3020 executes control programs stored in the storage medium 3026 using the CPU 3024 etc., and thereby, realizes various functions including an ultrasonic measurement according to the embodiment.

Specifically, the main body device 3020 transmits and applies an ultrasonic beam toward an in vivo tissue of a subject 3002 from the ultrasonic probe 3016 and receives reflected wave. Then, the received signals of the reflected wave are amplified and signal-processed, and thereby, measurement data on the in vivo structure of the subject 3002 may be generated. The measurement data contains images of respective modes of the so-called A-mode, B-mode, M-mode, and color Doppler. The measurement using ultrasonic wave is repeatedly executed at a predetermined cycle. The unit of measurement is referred to as “frame”.

The ultrasonic probe 3016 includes a plurality of ultrasonic transducers arranged therein. In the embodiment, a single row is used, however, a two-dimensional arrangement configuration including a plurality of rows may be used. Further, the ultrasonic probe 3016 is fixed to the neck of the subject 3002 in the opposed position in which the arrangement of the ultrasonic transducers may extend along the long-axis direction of a carotid artery (vessel) 3004 of the subject 3002, and the vessel function information is measured.

### Principle

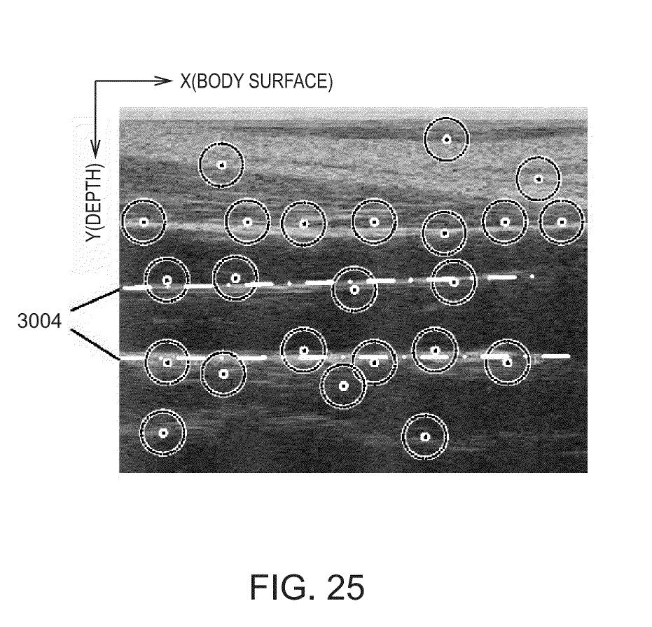

For measurement of the vessel function information, first, detection of a vessel position is performed. Specifically, as shown in FIG. 25, feature points in a B-mode image (center positions of dotted circles in FIG. 25) are extracted. The B-mode image shown in FIG. 25 is a sectional view of the vessel in the long-axis direction, and the X axis extends along the living body surface and the Y-axis extends in the depth direction from the living body surface. Note that, to facilitate understanding, the reduced number of feature points is shown in the respective drawings of FIG. 25 and the subsequent drawings, however, actually, more feature points than those shown in the drawings are extracted. Further, in FIG. 25, a position of the vessel wall of a real vessel 3004 is shown by dashed-dotted lines.

The section shape of the vessel 3004 in the long-axis direction is a shape with two straight lines nearly in parallel because the vessel walls appear. Further, in the B-mode image, many of the feature points appear in parts in which luminance changes such as muscle, tendons, and fat in addition to the vessel wall (specifically, an intima-adventitia boundary and a lumen-intima boundary). The reflectance of ultrasonic wave is higher in the position where the medium changes (in a sense, the boundary of medium), and the position with higher reflectance is represented in higher luminance in the B-mode image. Accordingly, the vessel wall, muscle, tendon, fat, etc. are different in medium from the surrounding tissues and the luminance changes in the parts, and the parts are extracted as feature points. One of the characteristics of the embodiment is to detect the vessel position using the position relationship of the feature points.

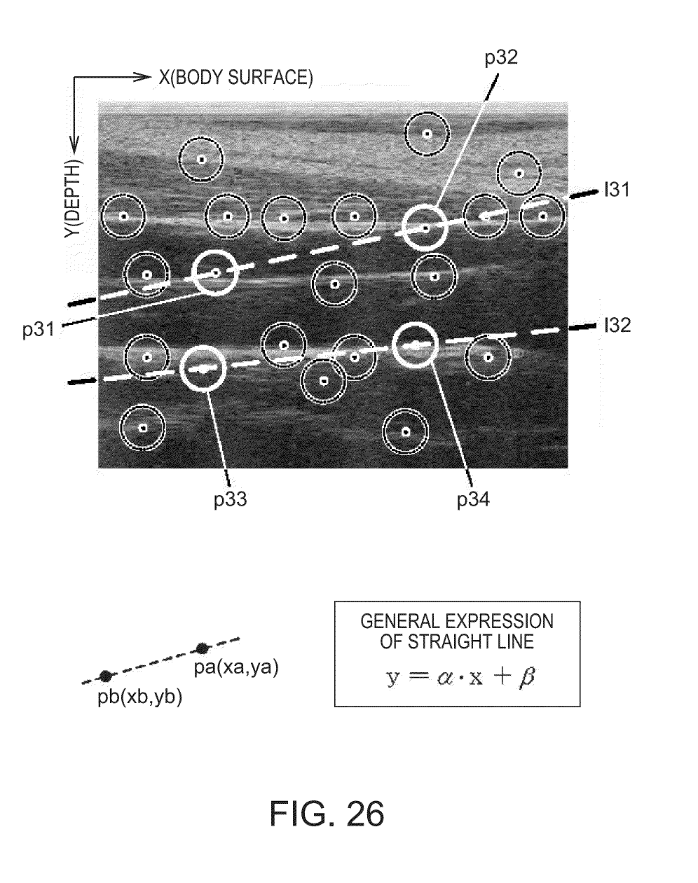

Specifically, as shown in FIG. 26, four feature points p31 to p34 (solid white circles in FIG. 26) are selected from the feature points in the B-mode image, and “set of feature points” as a combination of the four feature points is generated. The feature points p31 to p34 are selected to have a location position relationship along the section shape of the vessel 3004 in the long-axis direction. That is, the feature points p31 to p34 are selected so that a straight line 131 passing through the two feature points p31, p32 and a straight line 132 passing through the two feature points p33, p34 may be nearly in parallel to each other and the distance between the straight lines may be equal to or less than a predetermined distance that is regarded as a vessel diameter.

Hereinafter, the two straight lines 131, 132 defined by the four feature points p31 to p34 forming the set of feature points are referred to as “pair of straight lines” of the set of feature points. Note that, regarding the two straight lines 131, 132, the shallower one in the depth position is the straight line 131 and the deeper one is the straight line 132.

A straight line 1 passing through two feature points pa, pb is defined by parameters α, β obtained by generating simultaneous linear equations with two unknowns by substituting position coordinates pa (xa, ya) and pb (xb, yb) of the respective two feature points in a general expression given by the formula (4) and solving the simultaneous equations.

y=α·x+β (4)

Then, the set of feature points are evaluated on a criterion as to whether or not the pair of straight lines of the set of feature points are regarded as the position of the vessel wall. Specifically, as shown by the formula (5), evaluation values fi by a plurality of evaluation items are weighted by a coefficient ai and added, and a comprehensive evaluation value F is calculated. As will be described later, the evaluation value fi is obtained from a probability density function hi (xi) with a variable xi as shown in the formula (6).

$$\begin{matrix}F = {\sum }_{i} ( {a}_{i} \times {f}_{i} ) & ( 5 ) \\ {f}_{i} = {h}_{i} ( {x}_{i} ) & ( 6 )\end{matrix}$$

The evaluation values fi of the respective evaluation items correspond to probabilities (also referred to as “accuracy”) of the two straight lines of the pair of straight lines located in the position of the vessel wall. That is, the values are defined to be larger as the probabilities that the pair of straight lines are regarded as the vessel wall are higher. Further, whether or not the pair of straight lines are regarded as the vessel wall is determined using the comprehensive evaluation value F, and the vessel position is decided. In this regard, setting of the evaluation items on which importance is placed for determination may be changed by the weight coefficient ai.

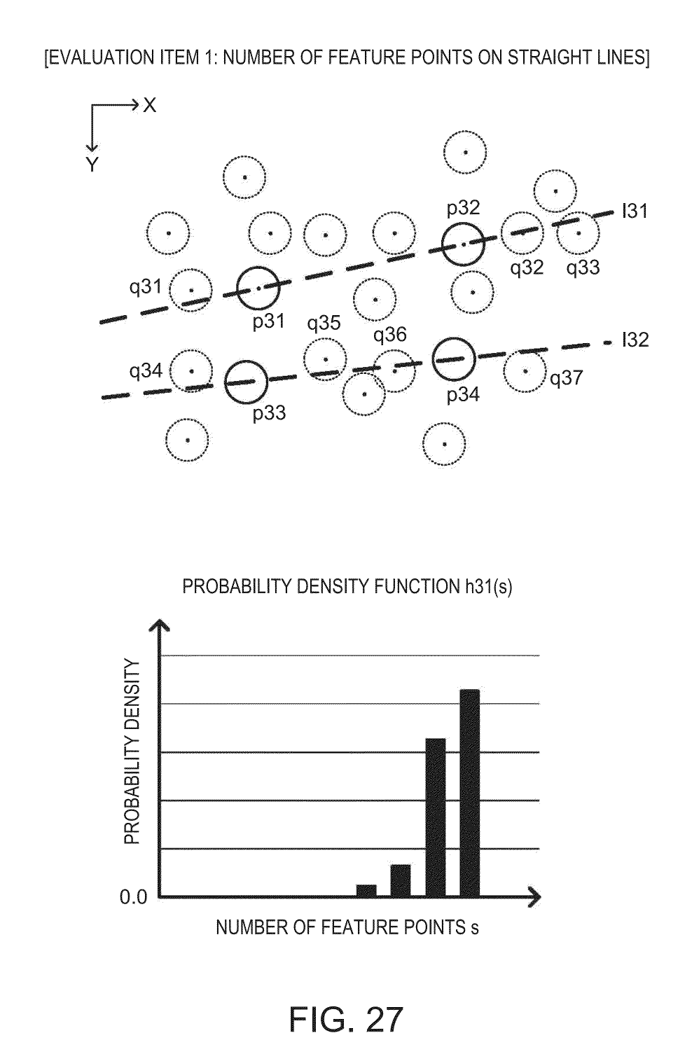

In the third embodiment, evaluations are made with respect to five evaluation items (first to fifth evaluation items). The first evaluation item is “number of feature points located on respective straight lines of pair of straight lines”. FIG. 27 is a diagram for explanation of an evaluation with respect to the first evaluation item in the third embodiment. The upper side of FIG. 27 shows the schematic locations of the feature points and the pair of straight lines in the B-mode image and the lower side of FIG. 27 shows a probability density function h31(s) of the number of feature points as an evaluation criterion. The valuable s is the number of feature points.

As shown in FIG. 27, the feature points located at the distances from the two straight lines 131, 132 equal to or less than a predetermined short distance are selected as feature points q (q31 to q37) on the respective straight lines 131, 132 of the pair of straight lines. Note that the feature points q selected here do not include the feature points p31 to p34 forming the set of feature points. Then, the probability density obtained from the probability density function h31(s) based on the number s of the selected feature points q is used as an evaluation value f31 of the first evaluation item.

The probability density function h31(s) shown in FIG. 27 is defined, with respect to many B-mode images containing sections in the long-axis direction of a vessel desired to be detected (e.g., a carotid artery) acquired in advance, by counting the numbers of feature points located on the vessel wall of the vessel. As described above, many feature points exist in the position of the vessel wall and there is a tendency that the numbers of the feature points are concentrated on predetermined numbers as shown by the probability density function h31(s).

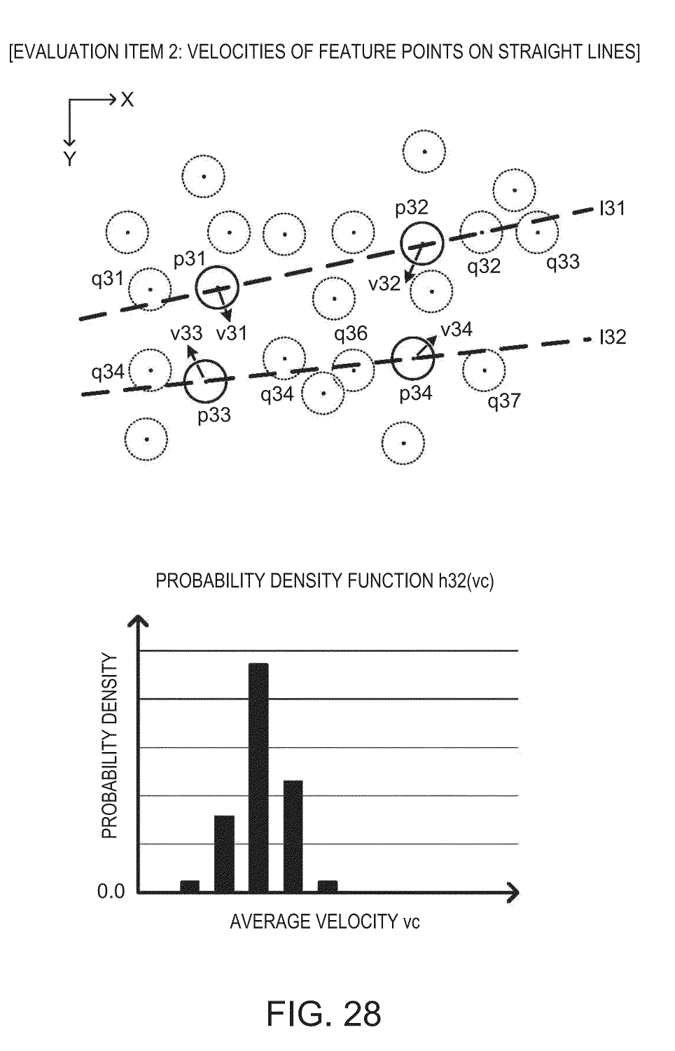

The second evaluation item is “displacement velocities of feature points located on respective straight lines of pair of straight lines”. The displacement velocity refers to a position change per unit time and a magnitude of the velocity (absolute value). FIG. 28 is a diagram for explanation of evaluations with respect to the second evaluation item in the third embodiment. The upper side of FIG. 28 shows the schematic locations of the feature points and the pair of straight lines in the B-mode image and the lower side of FIG. 28 shows a probability density function h32(vc) of the average displacement velocity of feature points as an evaluation criterion. The variable vc is an average velocity.

As shown in FIG. 28, the displacement velocities of the respective feature points q (q31 to q37) on the respective straight lines 131, 132 of the pair of straight lines are obtained, and average velocities vc as averages of the displacement velocities are obtained. For example, the displacement velocities of the respective feature points q are obtained by obtaining velocity vectors v of the feature points q and averaging the magnitudes of the velocity vectors v over a predetermined period (equal to or more than one heartbeat of a heartbeat period, about several seconds) using a gradient method utilizing spatial luminance gradients, block matching utilizing an image block having a predetermined size and containing the feature points as a template, or the like. Then, the probability density obtained from the probability density function h32(vc) in FIG. 28 based on the obtained average velocity vc is used as an evaluation value f32 of the second evaluation item.

The probability density function h32(vc) shown in FIG. 28 is defined, with respect to many B-mode images containing sections in the long-axis direction of a vessel desired to be detected (e.g., a carotid artery) acquired in advance, by obtaining average velocities of the respective feature points located on the vessel wall of the vessel. The vessel repeats generally isotropic constriction and dilatation according to the beats of the heart. That is, the magnitude of the displacement velocity of the feature point located on the vessel wall periodically changes in terms of the heartbeat period, and the average in one heartbeat period is nearly constant in any heartbeat period. Accordingly, there is a tendency that the average values of the magnitudes of the displacement velocities of one heartbeat period are concentrated on predetermined values as shown by the probability density function h32(vc).

Note that, as the displacement velocities of the respective feature points, velocity components in the depth direction (i.e., components of velocity vectors v in the depth direction) may be used. Further, not the displacement velocity, but acceleration may be used.

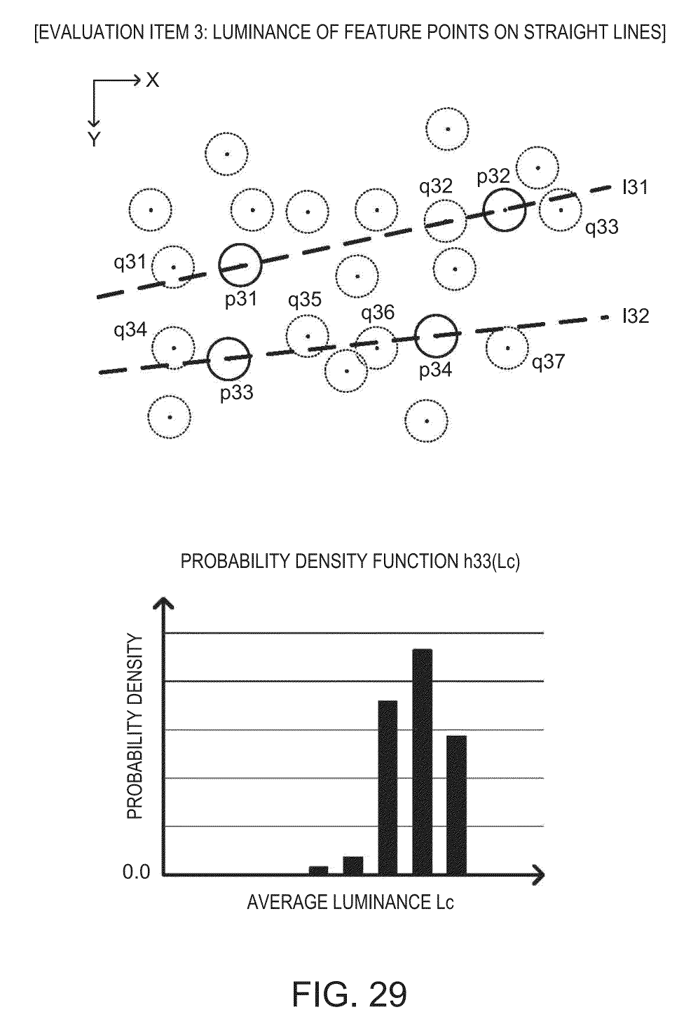

The third evaluation item is “luminance of feature points located on respective straight lines of pair of straight lines”. FIG. 29 is a diagram for explanation of evaluations with respect to the third evaluation item in the third embodiment. The upper side of FIG. 29 shows the schematic locations of the feature points and the pair of straight lines in the B-mode image and the lower side of FIG. 29 shows a probability density function h33(Lc) of the luminance as an evaluation criterion. The variable Lc is average luminance.

As shown in FIG. 29, average luminance Lc as averages of the luminance L of the respective feature points q (q31 to q37) on the respective straight lines 131, 132 of the pair of straight lines is obtained. Then, the probability density obtained from the probability density function h33(Lc) based on the obtained average luminance Lc is used as an evaluation value f33 of the third evaluation item.

The probability density function h33(Lc) shown in FIG. 29 is defined, with respect to many B-mode images containing sections in the long-axis direction of a vessel desired to be detected (e.g., a carotid artery) acquired in advance, by obtaining average values of luminance of feature points located on the vessel wall of the vessel. As described above, many feature points exist on the vessel wall, and there is a tendency that the average luminance of the feature points is concentrated on predetermined relatively high luminance as shown by the probability density function h33(Lc).

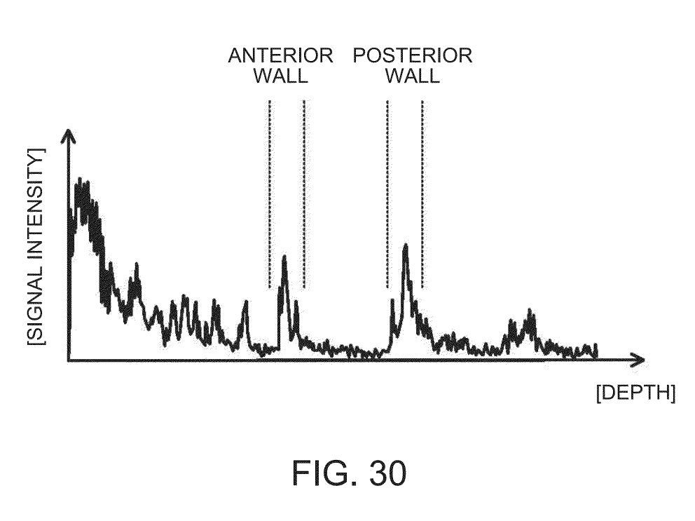

Note that, not the luminance of the feature points itself, but “gradient of luminance” may be used. That is, as shown in FIG. 30, in A-mode data (depth-signal intensity graph), signal intensity (i.e., luminance) largely changes in the depth position of the vessel wall. Thereby, as the gradients of luminance of the feature points q located on the straight lines 131, 132, gradients in the depth positions of the feature points q in the A-mode data (changes in signal intensity as seen in the depth direction) may be obtained, and the probability density may be obtained based on the average value of the gradients of luminance and used as the evaluation value of the third evaluation item. Further, as the gradients of luminance of the feature points q, differences between the luminance of the feature points q in the B-mode image and the luminance of pixels adjacent to the feature points q in the depth direction may be used.

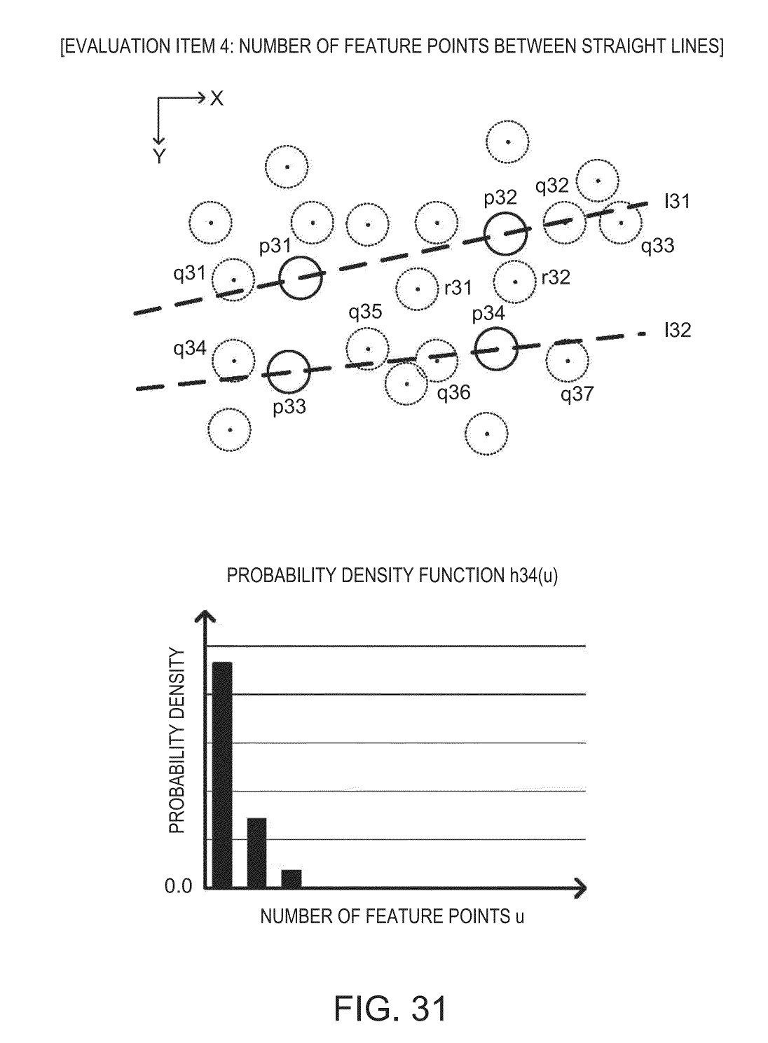

The fourth evaluation item is “number of feature points between straight lines”. FIG. 31 is a diagram for explanation of evaluations with respect to the fourth evaluation item. The upper side of FIG. 31 shows the schematic locations of the feature points and the pair of straight lines in the B-mode image and the lower side of FIG. 31 shows a probability density function h34(u) of the number of feature points as an evaluation criterion. The variable u is the number of feature points.

As shown in FIG. 31, feature points r (r31, r32) located between the straight lines 131, 132 of the pair of straight lines are selected. The feature points r selected here do not include the feature points p31 to p34 forming the set of feature points or the feature points q (q31 to q37) on the straight lines 131, 132 as the evaluation objects from the first evaluation item to the third evaluation item. Then, the probability density obtained from the probability density function h34(u) based on the number u of the selected feature points r is used as an evaluation value f34 of the fourth evaluation item.

The probability density function h34(u) shown in FIG. 31 is defined, with respect to many B-mode images containing sections in the long-axis direction of a vessel desired to be detected (e.g., a carotid artery) acquired in advance, by counting the numbers of feature points within the vessel (inside the vessel wall). The reflectance of ultrasonic wave by the vessel wall is higher, however, the reflectance by the blood within the vessel is extremely lower and the ultrasonic wave is hardly reflected, but transmitted. That is, there is a tendency that the numbers of feature points within the vessel are concentrated on predetermined numbers (values close to zero).

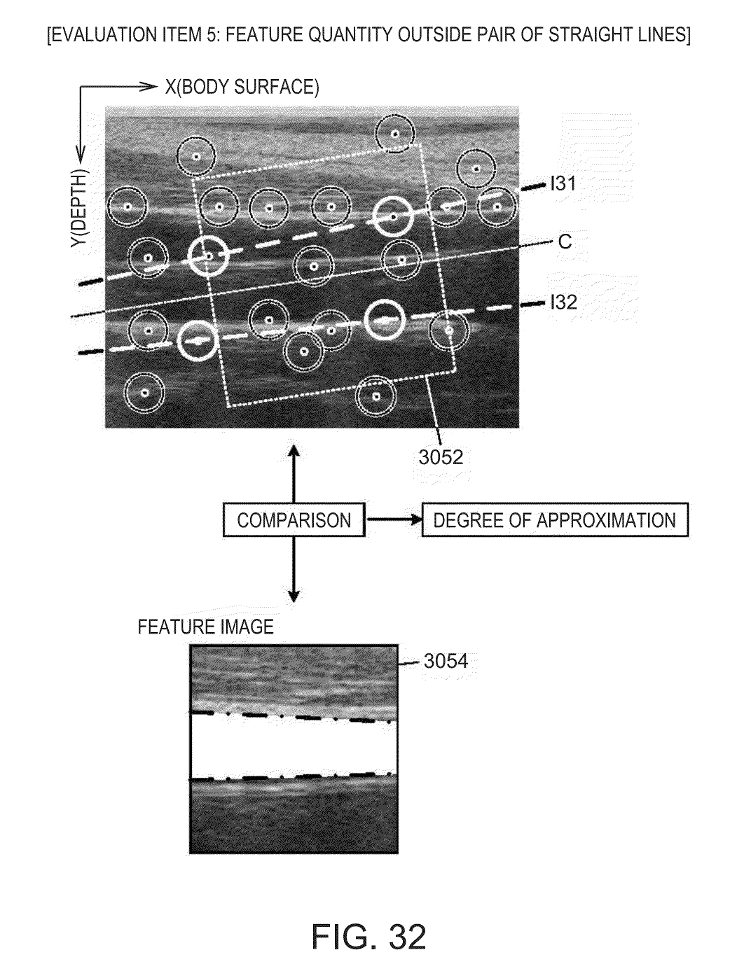

The fifth evaluation item is “feature quantity of external image of pair of straight lines”. FIG. 32 is a diagram for explanation of evaluations with respect to the fifth evaluation item in the third embodiment. In the evaluations of the fifth evaluation item, as shown by the upper side of FIG. 32, a partial image 3052 in a predetermined range containing the pair of straight lines is extracted as an evaluation object image from the B-mode image, feature quantity comparison processing between the partial image 3052 and a feature image 3054 prepared in advance is performed, and a degree of approximation of the images is calculated. The degree of approximation is used as an evaluation value f35 of the fifth evaluation item.

More specifically, the partial image 3052 is set as an external image of the pair of straight lines and a rectangle with the center line C between the straight lines 131, 132 crossing the image center in the lateral direction. Further, the partial image 3052 is a square image with the direction along the center line C of the straight lines 131, 132 as the lateral direction and the direction orthogonal to the center line C as the longitudinal direction, and the lengths in the longitudinal direction and the lateral direction are lengths based on the distance between the straight lines 131, 132 (e.g., three times the distance between the straight lines 131, 132).

The feature image 3054 is a B-mode image of the body tissues outside the section in the long-axis direction (above the anterior wall and below the posterior wall) of the vessel desired to be detected (e.g., a carotid artery) and the image in which the center line of the vessel in the long-axis direction crosses the image center in the lateral direction. Further, the relative position and relative size of the vessel wall (anterior wall and posterior wall) to the whole feature image 3054 are the same as the relationship between the pair of straight lines in the partial image 3052. Around the vessel, muscle fibers and groups of lymph nodes can exist as body tissues, and the feature image 3054 contains pattern components of the body tissues.

In the feature quantity comparison processing, the degree of approximation is calculated by a comparison calculation of the image parts formed by removing the part between the straight lines 131, 132 of the pair of straight lines from the partial image 3052, i.e., the image part above the straight line 131 and the image part below the straight line 132 as the image parts outside the pair of straight lines with the feature image 3054. In the calculation of the degree of approximation, for example, the degree of approximation may be obtained by comparison between the location relationships of the feature points in the images, distributions of luminance, texture information of the images, or the like using the so-called pattern matching or the like.

### Functional Configuration

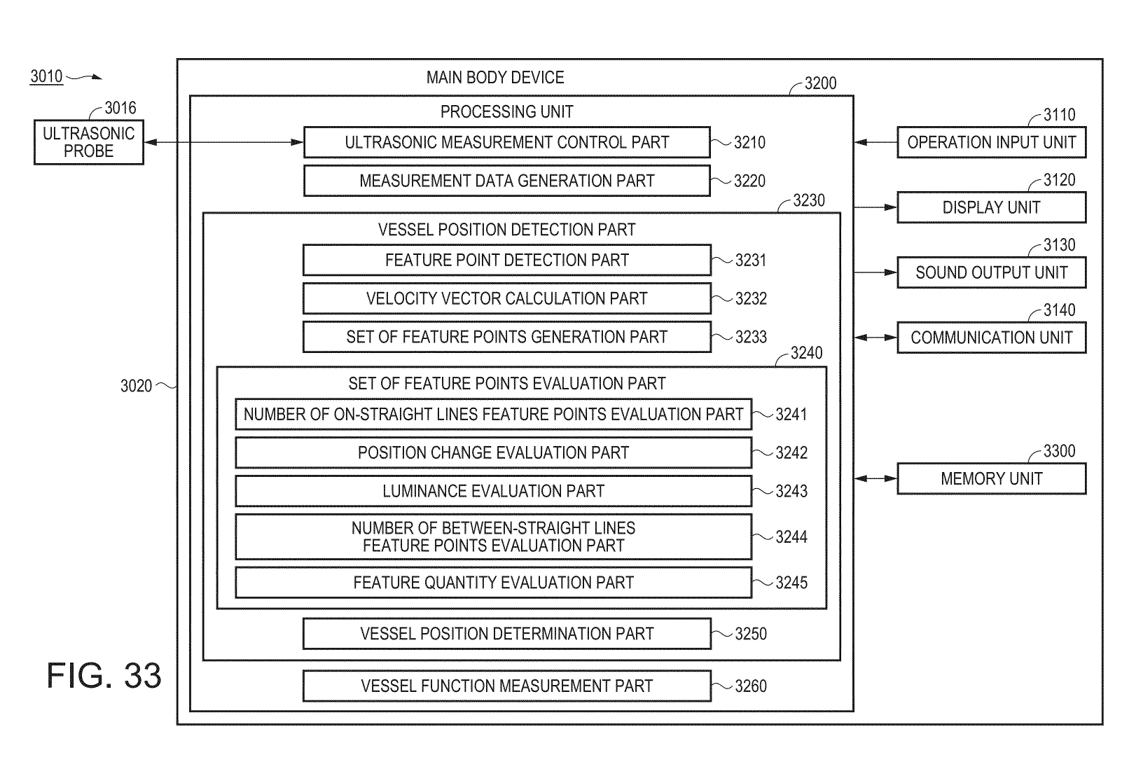

FIG. 33 is a functional configuration diagram of the ultrasonic measuring apparatus 3010 in the third embodiment. As shown in FIG. 33, the ultrasonic measuring apparatus 3010 includes the main body device 3020 and the ultrasonic probe 3016. The main body device 3020 includes an operation input unit 3110, a display unit 3120, a sound output unit 3130, a communication unit 3140, a processing unit 3200, and a memory unit 3300.

The operation input unit 3110 is realized by input devices including a button switch, a touch panel, various sensors or the like, and outputs an operation signal in response to the performed operation to the processing unit 3200. In FIG. 24, the touch panel 3012 and the keyboard 3014 correspond to the unit.

The display unit 3120 is realized by a display device such as an LCD (Liquid Crystal Display) and performs various kinds of display based on display signals from the processing unit 3200. In FIG. 24, the touch panel 3012 corresponds to the unit.

The sound output unit 3130 is realized by a sound output device such as a speaker and performs various kinds of sound output based on sound signals from the processing unit 3200.

The communication unit 3140 is realized by a wireless communication device such as a wireless LAN (Local Area Network) or Bluetooth (registered trademark) or a communication device such as a modem, a jack of a wire communication cable, or a control circuit, and connects to a given communication line and performs communication with an external device. In FIG. 24, the unit corresponds to a communication IC 3028 mounted on a control board 3022.

The processing unit 3200 is realized by a microprocessor such as a CPU (Central Processing Unit) or GPU (Graphics Processing Unit) or an electronic component such as an ASIC (Application Specific Integrated Circuit) or IC (Integrated Circuit) memory, and executes various kinds of calculation processing based on the programs and data stored in the memory unit 3300, the operation signal from the operation input unit 3110, etc. and controls the operation of the ultrasonic measuring apparatus 3010. Further, the processing unit 3200 has an ultrasonic measurement control part 3210, a measurement data generation part 3220, a vessel position detection part 3230, and a vessel function measurement part 3260.

The ultrasonic measurement control part 3210 controls transmission and reception of ultrasonic wave in the ultrasonic probe 3016. Specifically, the part allows the ultrasonic probe 3016 to transmit ultrasonic wave at transmission times at a predetermined cycle. Further, the part performs amplification of a signal of reflected wave of ultrasonic wave received in the ultrasonic probe 3016 etc.

The measurement data generation part 3220 generates measurement data containing image data of the respective modes of the A-mode, B-mode, and M-mode based on the received signals of the reflected wave by the ultrasonic probe 3016.

The vessel position detection part 3230 has a feature point detection part 3231, a velocity vector calculation part 3232, a set of feature points generation part 3233, a set of feature points evaluation part 3240, and a vessel position determination part 3250, and performs detection of the vessel position based on the measurement data generated by the measurement data generation part 3220.

The feature point detection part 3231 detects feature points in a B-mode image. In the detection of feature points, pixels that satisfy a predetermined condition are detected as feature points based on the luminance of the pixels, luminance differences between the pixels and the surrounding pixels of the pixels, or the like.

The velocity vector calculation part 3232 compares temporally adjacent B-mode images, and calculates velocity vectors (magnitudes and directions of velocities) of the respective feature points based on the amounts of movements and frame rates of the feature points.

The set of feature points generation part 3233 generates a set of feature points including four feature points selected from the detected feature points. In this regard, the four feature points p31 to p34 are selected so that the straight line 131 passing through the two feature points p31, p32 and the straight line 132 passing through the two feature points p33, p34 may be nearly in parallel and the distance between the straight lines may be equal to or less than a predetermined distance. The straight lines 131, 132 are calculated by obtaining the parameters α, β in the definitional equation of straight line (4). To obtain the straight lines 131, 132 is to set the pair of straight lines corresponding to the section shape in the long-axis direction corresponding to the long-axis section of the vessel.

The set of feature points evaluation part 3240 has a number of on-straight lines feature points evaluation part 3241, a position change evaluation part 3242, a luminance evaluation part 3243, a number of between-straight lines feature points evaluation part 3244, and a feature quantity evaluation part 3245, and the set of feature points are evaluated on the criterion as to whether or not the pair of straight lines of the set of feature points is regarded as the position of the vessel wall. Specifically, as shown in the above formula (5), the item evaluation values fi obtained with respect to each of the plurality of evaluation items are multiplied by the weight coefficient ai and added, and thereby, the comprehensive evaluation value F is calculated.

The number of on-straight lines feature points evaluation part 3241 performs an evaluation based on “number of feature points located on respective straight lines of pair of straight lines” as the first evaluation item. That is, the number s of the feature points q located on the respective straight lines 131, 132 in the B-mode image, and the probability density obtained from the probability density function h31(s) based on the obtained number s of feature points is used as the evaluation value f31 of the first evaluation item (see FIG. 27).

The position change evaluation part 3242 performs an evaluation based on “position changes of feature points on respective straight lines of pair of straight lines” as the second evaluation item. That is, averages of the displacement velocities (position changes per unit time) of the respective feature points q on the respective straight lines 131, 132 in the B-mode image over a predetermined period (e.g., equal to or more than one heartbeat of a heartbeat period, about several seconds) are used as displacement velocities vi of the feature points, and an average velocity vc as an average of the displacement velocities vi of the respective feature points q is obtained. Then, the probability density obtained from the probability density function h32(vc) based on the obtained average velocity vc is used as the evaluation value f32 of the second evaluation item (see FIG. 28).

The luminance evaluation part 3243 performs an evaluation based on “luminance of feature points on respective straight lines of pair of straight lines” as the third evaluation item. That is, an average value of luminance L of the respective feature points q located on the straight lines 131, 132 in the B-mode image is obtained, and the probability density obtained from the probability density function h33(Lc) based on the obtained average luminance Lc is used as the evaluation value f33 of the third evaluation item (see FIG. 29).

The number of between-straight lines feature points evaluation part 3244 performs an evaluation based on “number of feature points between straight lines” as the fourth evaluation item. That is, the number u of the feature points r located between the straight lines 131, 132 in the B-mode image is obtained, and the probability density obtained from the probability density function h34(u) based on the obtained number u of feature points is used as the evaluation value f34 of the fourth evaluation item (see FIG. 31).

The feature quantity evaluation part 3245 performs an evaluation based on “feature quantity of external image of pair of straight lines” as the fifth evaluation item. That is, the degree of approximation of the images is calculated by extracting the partial image 3052 containing the pair of straight lines in the B-mode image so that the relative position and relative size of the pair of straight lines may have the same relationship with the whole image as the vessel wall in the feature image 3054 and comparing the image with the feature image 3054 prepared in advance, and the calculated degree of approximation is used as the evaluation value f35 of the fifth evaluation item (see FIG. 32).

The vessel position determination part 3250 determines the vessel position using the evaluation result with respect to the set of feature points by the set of feature positions evaluation part 3240. Specifically, existence of the vessel wall in the position of the respective straight lines 131, 132 by the set of feature points having the maximum comprehensive evaluation value F is determined, the average distance between the straight lines 131, 132 corresponding to the center line C of the straight lines 131, 132 and the radius R of the vessel is obtained, and the vessel position is decided.

In this regard, in order to determine the vessel position with higher accuracy, the feature points on the respective straight lines 131, 132 in the B-mode image may be reselected, the respective straight lines 131, 132 may be recalculated using e.g. the least-square method based on the reselected feature points, and the center line C and the radius R may be decided based on the recalculated straight lines 131, 132.

The vessel function measurement part 3260 performs measurements of given vessel function information. Specifically, the part performs measurements of vessel function information of the measurement of the vessel diameter, IMT, etc. of the vessel specified by the detected vessel position, measurements of the pulse wave propagation velocity and the hardness index value of the vessel wall, the estimation calculation of blood pressure from vessel diameter fluctuations by tracking the vessel anterior wall and the vessel posterior wall, and the calculation of the pulse rate.

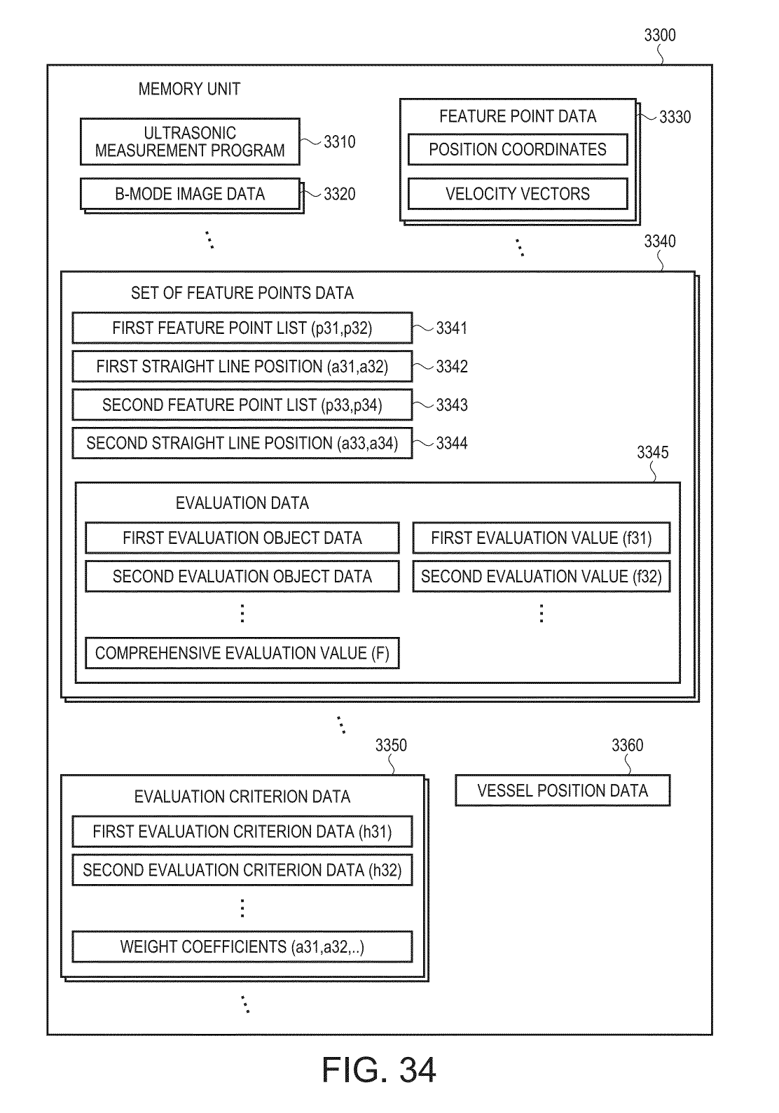

The memory unit 3300 is realized by a memory device such as ROM, RAM, or hard disk, stores programs, data, etc. for integrated control of the ultrasonic measuring apparatus 3010 by the processing unit 3200 and used as a work area of the processing unit 3200, and calculation results executed by the processing unit 3200, operation data from the operation input unit 3110, etc., are temporarily stored therein. In FIG. 24, the part corresponds to the storage medium 3026 mounted on the control board 3022. In the embodiment, as shown in FIG. 34, an ultrasonic measurement program 3310, B-mode image data 3320, feature point data 3330, set of feature points data 3340, evaluation criterion data 3350, and vessel position data 3360 are stored in the memory unit 3300.

The B-mode image data 3320 stores B-mode images generated with respect to each measurement frame associated with frame IDs.

The feature point data 3330 is generated with respect to each detected feature point and stores position coordinates and velocity vectors in the B-mode images in the respective frames.

The set of feature points data 3340 is generated with respect to each set of feature points. Since one pair of straight lines are defined by a set of feature points, the set of feature points data 3340 contains a first feature point list 3341 of the position coordinates of the feature points p31, p32 used for obtaining one straight line 131 of the pair of straight lines, a first line position 3342 defining the one straight line 131, a second feature point list 3343 of the position coordinates of the feature points p33, p34 used for obtaining the other straight line 132, and a second line position 3344 defining the other straight line 132. Further, the set of feature points data 3340 stores evaluation data 3345 used for the evaluation of the set of feature points (in other words, may be referred to as “evaluation of pair of straight lines”). The first line position 3342 and the second line position 3344 store the parameters α, β in the corresponding definitional equation of straight line (4). The evaluation data 3345 stores evaluation object data and evaluation values for the respective plurality of evaluation items and comprehensive evaluation values.

The evaluation criterion data 3350 stores evaluation criteria (probability density functions h31 to h34, the feature image 3054, etc.) for the respective plurality of evaluation items and the weight coefficients a31 to a35.

The vessel position data 3360 is data of the detected vessel position and stores e.g., the position coordinates of the center line C of the long-axis section and the radius R of the vessel.

### Flow of Processing

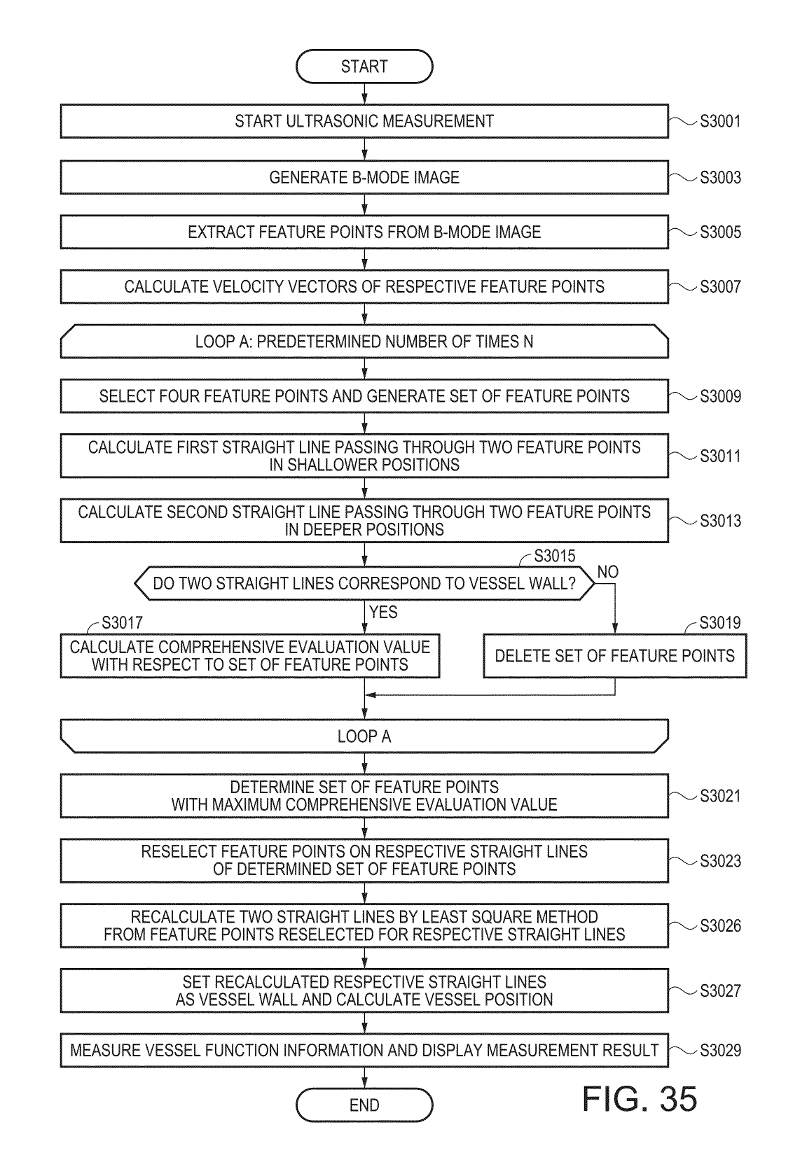

FIG. 35 is a flowchart for explanation of the ultrasonic measuring processing in the third embodiment. The processing is realized by the processing unit 3200 executing the ultrasonic measurement program 3310.

The processing unit 3200 first starts an ultrasonic measurement using the ultrasonic probe 3016 (step S3001). Then, the measurement data generation part 3220 generates a B-mode image based on received signals of ultrasonic reflected wave by the ultrasonic probe 3016 (step S3003). Subsequently, the feature point detection part 3231 extracts feature points from the B-mode image (step S3005). Then, the velocity vector calculation part 3232 calculates velocity vectors of the respective extracted feature points (step S3007).

Then, the processing of loop A is repeated in a predetermined number of times. In the loop A, the set of feature points generation part 3233 selects four feature points from the previously extracted feature points and generates a set of feature points (step S3009). Then, the part selects two feature points p31, p32 in the shallower depth positions of the selected four feature points, obtains the parameters α, β of the straight line passing through the points, and calculates the first straight line 131 (step S3011). Further, the part selects two feature points p33, p34 in the deeper depth positions of the selected four feature points, obtains the parameters α, β of the straight line passing through the points, and calculates the second straight line 132 (step S3013).

Then, whether or not the pair of straight lines of the calculated two straight lines 131, 132 satisfy a predetermined vessel section condition regarded as a corresponding shape of the section of the vessel in the long-axis direction. The vessel section condition is e.g., “the two straight lines 131, 132 are nearly in parallel (the parameters α are nearly equal) and the distance between the straight lines 131, 132 is equal to or less than a predetermined distance”. If the pair of straight lines do not satisfy the vessel section condition (step 53015: NO), the points are not employed as the set of feature points and deleted (step S3019). On the other hand, if the pair of straight lines satisfy the vessel section condition (step S3015: YES), the set of feature points are employed and the set of feature points evaluation part 3240 calculates a comprehensive evaluation value F of the set of feature points (step S3017).

For calculation of the comprehensive evaluation value F, the number of on-straight lines feature points evaluation part 3241 obtains the number s of feature points located on the respective straight lines 131, 132 in the B-mode image and probability density obtained from the probability density function h31(s) based on the obtained number s of feature points is used as the evaluation value f31 of the first evaluation item. Further, the position change evaluation part 3242 obtains an average velocity vc as an average of displacement velocities Vi of the respective feature points q located on the respective straight lines 131, 132 in the B-mode image and probability density obtained from the probability density function h32(vc) based on the obtained average velocity vc is used as the evaluation value f32 of the second evaluation item. Furthermore, the luminance evaluation part 3243 obtains an average value of luminance L of the respective feature points q located on the respective straight lines 131, 132 in the B-mode image and probability density obtained from the probability density function h33(Lc) based on the obtained average luminance Lc is used as the evaluation value f33 of the third evaluation item. Further, the number of between-straight lines feature points evaluation part 3244 obtains the number u of the feature points r located between the straight lines 131, 132 in the B-mode image and probability density obtained from the probability density function h34(u) based on the obtained number u of feature points is used as the evaluation value f34 of the fourth evaluation item. Furthermore, the feature quantity evaluation part 3245 compares the partial image 3052 around an assumed circle 3050 in the B-mode image with the feature image 3054 and calculates a degree of approximation of the images, and the calculated degree of approximation is used as the evaluation value f35 of the fifth evaluation item. Then, the set of feature point evaluation part 3240 multiplies the calculated evaluation values f31 to f35 of the respective evaluation items by the corresponding weight coefficients a31 to a35 and adds up them, and thereby, calculates a comprehensive evaluation value F. The loop A is performed in the above described manner.

When the processing of the loop A at the predetermined number of times is ended, the vessel position determination part 3250 determines the set of feature points having the maximum comprehensive evaluation value F from all sets of feature points (step S3021). Then, the plurality of feature points located on the respective straight lines 131, 132 of the pair of straight lines by the determined set of feature points are reselected (step S3023), the parameters of the respective straight lines 131, 132 are recalculated by the least-square method using the positions of the reselected feature points, and the position of the respective straight lines 131, 132 is recalculated (step S3025). Then, the center line C and the radius R in the long-axis section of the vessel are decided from the recalculated position of the respective straight lines 131, 132 and the vessel position is obtained (step S3027).

Then, the vessel function measurement part 3260 performs a measurement of given vessel function information using the transmission and reception results of ultrasonic wave by the ultrasonic probe 3016, and stores and displays the measured vessel (step S3029). This is the end of the ultrasonic measurement processing.

### Advantages

As described above, according to the third embodiment, the combination of the feature points such that the positions of the feature points in the ultrasonic image may have a location relationship along the section shape of the vessel in the long-axis direction is selected, and the position of the vessel is determined using the evaluation result of the selected combination. In the ultrasonic image containing the section of the vessel in the long-axis direction, there is a characteristic that many feature points appear along the pair of straight lines as the section shape of the vessel. Thereby, a new technology of detecting the position of the vessel from the location relationship of the positions of the feature points in the ultrasonic image may be realized.

Note that, in the third embodiment, four feature points p31 to p34 form the set of feature points, however, five or more feature points may form the set of feature points. That is, as the straight lines 131, 132 corresponding to the position of the vessel wall, straight lines passing through three or more feature points may be obtained. Further, not the straight lines 131, 132, but curved lines allowing a little curve having a curvature equal to or less than a fixed value may be employed. Furthermore, a thickness may be additionally defined for the straight lines 131, 132 and the straight lines 131, 132 may be regarded as elongated rectangles. The additional definition of thickness may include curved lines allowing curvature to some degree.

Further, as the evaluation items for evaluation of the set of feature points, the five evaluation items are explained as an example, however, it is not necessary to use all evaluation items for determination of the comprehensive evaluation value F. Of the five evaluation items, one or more selected evaluation items may be used for determination of the comprehensive evaluation value F. Or, other evaluation items may be used.

As described above, the embodiments of the invention are explained in detail, however, a person skilled in the art could readily understand that many modifications may be made without substantially departing from the new matter and the effects of the invention. Therefore, such modified examples may fall within the range of the invention.

The entire disclosure of Japanese Patent Application No. 2014-075750 filed on Apr. 1, 2014 and No. 2014-257683 filed on Dec. 19, 2014 are expressly incorporated by reference herein.

1-20. (canceled)

21. An ultrasonic measuring apparatus comprising:
an ultrasonic measurement unit that transmits and receives ultrasonic wave with respect to a vessel and acquires an ultrasonic image containing a section in a short-axis direction of the vessel;
a feature point extraction unit that extracts feature points from the ultrasonic image;
a combination selection unit that selects a combination of feature points in which positions of the feature points have a location relationship along a section shape of the vessel in the short-axis direction; and
a position determination unit that determines a position of the vessel using the combination.

22. The ultrasonic measuring apparatus according to claim 21, wherein a contour position of a shape corresponding to the section of the vessel in the short-axis direction is estimated based on the location relationship with respect to the combination, and the position of the vessel is determined by calculation of probabilities of the contour position representing a position of a vessel wall of the vessel based on the contour position and the feature points.

23. The ultrasonic measuring apparatus according to claim 22, wherein a first of the probabilities is calculated using a number of the feature points located along the contour position.

24. The ultrasonic measuring apparatus according to claim 22, wherein a second of the probabilities is calculated using position changes of the feature points located along the contour position.

25. The ultrasonic measuring apparatus according to claim 22, wherein a third of the probabilities is calculated using luminance of the feature points located along the contour position.

26. The ultrasonic measuring apparatus according to claim 22, wherein a fourth of the probabilities is calculated using a number of the feature points located inside the contour position.

27. The ultrasonic measuring apparatus according to claim 22, wherein a fifth of the probabilities is calculated by comparison between a predetermined feature image that may be contained outside the vessel and an external image part of the contour position of the ultrasonic image.

28. The ultrasonic measuring apparatus according to claim 21, wherein the position determination unit determines a scanning line passing through a center of the vessel of a plurality of scanning lines with respect to transmission and reception of the ultrasonic wave using the combination.

29. The ultrasonic measuring apparatus according to claim 28, wherein the combination selection unit selects the combination of feature points in terms of scanning lines.

30. The ultrasonic measuring apparatus according to claim 29, wherein the feature point extraction unit extracts adventitia positions and lumen-intima boundary positions with respect to an anterior wall and a posterior wall as feature points, and the position determination unit evaluates luminance of the respective feature points contained in the combination by a predetermined evaluation calculation, and specifies a scanning line with respect to the combination receiving a highest evaluation as a scanning line passing through the center of the vessel.

31. The ultrasonic measuring apparatus according to claim 21, wherein the vessel is an artery.

32. The ultrasonic measuring apparatus according to claim 21, further comprising a measurement unit that measures a predetermined vessel function of the vessel detected by the position determination unit.

33. An ultrasonic measuring apparatus comprising:
an ultrasonic measurement unit that transmits and receives ultrasonic wave with respect to a vessel and acquires an ultrasonic image containing a section in a long-axis direction of the vessel;
a feature point extraction unit that extracts feature points from the ultrasonic image;
a combination selection unit that selects a combination of feature points in which positions of the feature points have a location relationship along a section shape of the vessel in the long-axis direction; and
a position determination unit that determines a position of the vessel using the combination.

34. The ultrasonic measuring apparatus according to claim 33, wherein a pair of straight lines corresponding to a section shape of the vessel in the long-axis direction is set in the ultrasonic image based on the location relationship with respect to the combination, probabilities of the pair of straight lines representing a position of a vessel wall of the vessel are calculated based on the pair of straight lines and the feature points, and the position of the vessel is determined using the probabilities and the combination.

35. The ultrasonic measuring apparatus according to claim 34, wherein the calculation of the probabilities includes a calculation of a first of the probabilities using a number of the feature points located along the pair of straight lines.

36. The ultrasonic measuring apparatus according to claim 34, wherein the calculation of the probabilities includes a calculation of a second of the probabilities using position changes of the feature points located along the pair of straight lines.

37. The ultrasonic measuring apparatus according to claim 34, wherein the calculation of the probabilities includes a calculation of a third of the probabilities using luminance of the feature points located along the pair of straight lines.

38. The ultrasonic measuring apparatus according to claim 34, wherein the calculation of the probabilities includes a calculation of a fourth of the probabilities using a number of the feature points located between the pair of straight lines.

39. The ultrasonic measuring apparatus according to claim 34, wherein the calculation of the probabilities includes a calculation of a fifth of the probabilities by comparison between a predetermined feature image that may be contained outside the vessel and an external image part of the pair of straight lines of the ultrasonic image.

40. The ultrasonic measuring apparatus according to claim 33, further comprising a measurement unit that measures a predetermined vessel function of the vessel detected by the position determination unit.

41. An ultrasonic measuring method of determining a vessel position from an ultrasonic image containing a section of a vessel in a short-axis direction or a long-axis direction using a computer, comprising;
extracting feature points from the ultrasonic image;
selecting a combination of feature points in which positions of the feature points have a location relationship along a section shape of the vessel in the short-axis direction or the long-axis direction; and
determining a position of the vessel using the combination.
